(19) United States
(12) Patent Application Publication
(10) Pub. No.: US 2010/0267749 A1
(43) Pub. Date: Oct. 21, 2010

(54) TRISUBSTITUTED FUROPYRIMIDINES AND USE THEREOF

(75) Inventors: Thomas Lampe, Dusseldorf (DE); Raimund Kast, Wuppertal (DE); Friederike Stoll, Dusseldorf (DE); Joachim Schuhmacher, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/743,227

(22) PCT Filed: Nov. 3, 2008
(86) PCT No.: PCT/EP2008/009237

§ 371 Date: May 14, 2010

(30) Foreign Application Priority Data
Nov. 16, 2007 (DE) 10 2007 054 786.4

(51) Int. Cl. A61K 31/519; C07D 487/04; A61P 7/02; A61P 9/10; A61P 9/12; A61P 11/00

(52) U.S. Cl. 514/260.1; 544/250

(57) ABSTRACT

The invention relates to the novel 4,5,6-trisubstituted furo[2,3-d]pyrimidine derivatives of formula (I), to methods for their production, their use in the treatment and/or prophylaxis of diseases and their use in the production of drugs for the treatment and/or prophylaxis of diseases, especially for the treatment and/or prophylaxis of cardiovascular diseases.

The present application relates to novel 4,5,6-trisubstituted furo[2,3-d]pyrimidine derivatives, to processes for their preparation, to their use for the treatment and/or prophylaxis of diseases and to their use for preparing medicaments for the treatment and/or prophylaxis of diseases, especially for the treatment and/or prophylaxis of cardio-vascular diseases.

Prostacyclin (PGI₂) belongs to the class of bioactive prostaglandins, which are derivatives of arachidonic acid. PGI₂ is the main product of arachidonic acid metabolism in endothelial cells and is a potent vasodilator and inhibitor of platelet aggregation. PGI₂ is the physiological antagonist of thromboxane A₂ (TxA₂), a strong vasoconstrictor and stimulator of platelet aggregation, and thus contributes to the maintenance of vascular homeostasis. A drop in PGI₂ levels is presumed to be partly responsible for the development of various cardiovascular diseases [Dusting, G. J. et al., Pharmac. Ther. 1990, 48: 323-344; Vane, J. et al., Eur. J. Vasc. Endovasc. Surg. 2003, 26: 571-578]. After release of arachidonic acid from phospholipids via phospholipases A₂, PGI₂ is synthesized by cyclooxygenases and then by PGI₂-synthase. PGI₂ is not stored, but is released immediately after synthesis, exerting its effects locally. PGI₂ is an unstable molecule, which is transformed rapidly (half-life approx. 3 minutes) and non-enzymatically, to an inactive metabolite, 6-keto-prostaglandin-F1alpha [Dusting, G. J. et al., Pharmac. Ther. 1990, 48: 323-344].

The biological effects of PGI₂ occur through binding to a membrane-bound receptor, called the prostacyclin receptor or IP receptor [Narumiya, S. et al., Physiol. Rev. 1999, 79: 1193-1226]. The IP receptor is one of the G-protein—Coupled receptors, which are characterized by seven transmembrane domains. In addition to the human IP receptor, prostacyclin receptors from rat and mouse have also been cloned [Vane, J. et al., Eur. J. Vasc. Endovasc. Surg. 2003, 26: 571-578]. In smooth muscle cells, activation of the IP receptor leads to stimulation of adenylate cyclase, which catalyzes the formation of cAMP from ATP. Increase in the intracellular cAMP concentration is responsible for prostacyclin-induced vasodilation and for inhibition of platelet aggregation. In addition to the vasoactive properties, anti-proliferative effects [Schroer, K. et al., Agents Actions Suppl. 1997, 48: 63-91; Kothapalli, D. et al., Mol. Pharmacol. 2003, 64: 249-258; Planchon, P. et al., Life Sci. 1995, 57: 1233-1240] and anti-arteriosclerotic effects [Rudic, R. D. et al., Circ. Res. 2005, 96: 1240-1247; Egan K. M. et al., Science 2004, 114: 784-794] have also been described for PGI₂. Furthermore, PGI₂ also inhibits the formation of metastases [Schneider, M. R. et al., Cancer Metastasis Rev. 1994, 13: 349-64]. It is unclear whether these effects are due to stimulation of cAMP formation or to IP receptor-mediated activation of other signal transduction pathways in the respective target cell [Wise, H. et al. TIPS 1996, 17: 17-21], such as the phosphoinositide cascade, and of potassium channels.

Although the effects of PGI₂ are on the whole of benefit therapeutically, clinical application of PGI₂ is severely restricted by its chemical and metabolic instability. It has been possible to make available PGI₂ analogs that are more stable, for example iloprost [Badesch, D. B. et al., J. Am. Coll. Cardiol. 2004, 43: 56S-61S] and treprostinil [Chattaraj, S. C., Curr. Opion. Invest. Drugs 2002, 3: 582-586], but these compounds still have a very short time of action. Moreover, the substances can only be administered to the patient via complicated routes of administration, e.g. by continuous infusion, subcutaneously or via repeated inhalations. These routes of administration can also have additional side-effects, for example infections or pain at the site of injection. The use of beraprost, which to date is the only PGI₂ derivative available for oral administration to patients [Barst, R. J. et al., J. Am. Coll. Cardiol. 2003, 41: 2119-2125], is once again limited by its short time of action.

It is an object of the present inventon to provide novel substances which act as chemically and metabolically stable, orally available activators of the IP receptor and are thus suitable for treating disorders, in particular cardiovascular disorders.

WO 03/018589 discloses 4-aminofuro[2,3-d]pyrimidines as adenosine kinase inhibitors for treating cardiovascular disorders. Furthermore, WO 2007/079861 and WO 2007/079862 describe 4-amino-, 4-oxy- or 4-thio-substituted 5,6-diphenylfuro[2,3-d]-pyrimidine derivatives and their use for treating cardiovascular disorders. Furo[2,3-d]-pyrimidines substituted in the 5- and/or 6-position by alkyl- and/or alkenyl radicals and their use for the treatment of various disorders are claimed in DE 1 817 146, WO 03/022852, WO 03/080064, WO 2005/092896, WO 2005/121149 and WO 2006/004658.

The present invention provides compounds of the general formula (I)

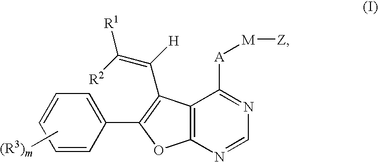

in which
R¹ is (C₁-C₆)-alkyl or a group of the formula —C(═O)—R^{1A} or —CH(OH)—R^{1B} in which R^{1A} represents (C₁-C₆)-alkyl, hydroxyl, (C₁-C₆)-alkoxy, (C₂-C₆)-alkenyloxy, amino, mono-(C₁-C₆)-alkylamino or mono-(C₂-C₆)-alkenylamino and
R^{1B} represents (C₁-C₆)-alkyl,
R² is hydrogen or (C₁-C₄)-alkyl,
R³ is a substituent selected from the group consisting of halogen, cyano, nitro, (C₁-C₆)-alkyl, (C₂-C₆)-alkenyl, (C₂-C₄)-alkynyl, (C₃-C₇)-Cycloalkyl, (C₄-C₇)-Cycloalkenyl, (C₁-C₆)-alkoxy, trifluoromethyl, trifluoromethoxy, (C₁-C₆)-alkylthio, (C₁-C₆)-acyl, amino, mono-(C₁-C₆)-alkylamino, di-(C₁-C₆)-alkylamino and (C₁-C₆)-acylamino,
where (C₁-C₆)-alkyl and (C₁-C₆)-alkoxy for their part may each be substituted by cyano, hydroxyl, (C₁-C₄)-alkoxy, (C₁-C₄)-alkylthio, amino, mono- or di-(C₁-C₄)-alkylamino, m is the number 0, 1 or 2,
where, if two substituents R³ are present, their meanings may be identical or different, A is O or N—R⁴, where
R⁴ represents hydrogen, (C₁-C₆)-alkyl, (C₃-C₇)-Cycloalkyl or (C₄-C₇)-Cycloalkenyl,
M is a group of the formula

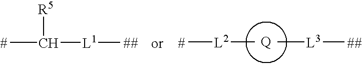

where
# represents the point of attachment to group A and
## represents the point of attachment to group Z,
R⁵ represents hydrogen or (C₁-C₄)-alkyl, which may be substituted by hydroxyl or amino,
L¹ represents (C₁-C₇)-alkanediyl or (C₂-C₇)-alkenediyl which may be mono- or disubstituted by fluorine, or represents a group of the formula *-L^{1A}-V-L^{1B}-** in which
* denotes the point of attachment to the group —CHR⁵,
** denotes the point of attachment to group Z,
L^{A} denotes (C₁-C₅)-alkanediyl which may be mono- or disubstituted by identical or different substituents from the group consisting of (C₁-C₄)-alkyl and (C₁-C₄)-alkoxy,
L^{1B} denotes a bond or (C₁-C₃)-alkanediyl, which may be mono- or disubstituted by fluorine, and
V denotes O or N—R⁶ where
R⁶ represents hydrogen, (C₁-C₆)-alkyl or (C₃-C₇)-Cycloalkyl,
L² represents a bond or (C₁-C₄)-alkanediyl,
L³ represents (C₁-C₄)-alkanediyl which may be mono- or disubstituted by fluorine and in which a methylene group may be replaced by O or N—R⁷, where
R⁷ denotes hydrogen, (C₁-C₆)-alkyl or (C₃-C₇)-Cycloalkyl, or represents (C₂-C₄)-alkenediyl, and
Q represents (C₃-C₇)-Cycloalkyl, (C₄-C₇)-Cycloalkenyl, phenyl, 5- to 7-membered heterocyclyl or 5- or 6-membered heteroaryl, each of which may be substituted up to two times by identical or different radicals selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino, where (C₁-C₄)-alkyl for its part may be substituted by hydroxyl, (C₁-C₄)-alkoxy, amino, mono-(C₁-C₄)-alkylamino or di-(C₁-C₄)-alkylamino, and
Z is a group of the formula

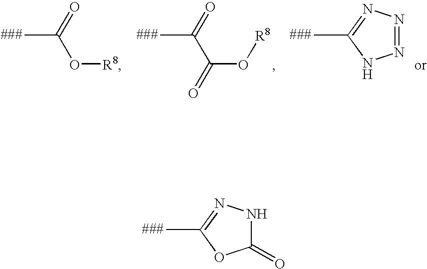

where
### represents the point of attachment to group L¹ or L³ and
R⁸ represents hydrogen or (C₁-C₄)-alkyl,
and their salts, solvates and solvates of the salts.

Compounds according to the invention are the compounds of the formula (I) and the salts, solvates and solvates of the salts thereof, the compounds of the formulae mentioned below encompassed by formula (I) and the salts, solvates and solvates of the salts thereof, and also the compounds encompassed by formula (I) and mentioned below as working examples, and the salts, solvates and solvates of the salts thereof, provided the compounds encompassed by formula (I) and mentioned below are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

If the compounds of the invention may occur in tautomeric forms, the present invention encompasses all tautomeric forms.

Salts which are preferred for the purposes of the present invention are physiologically acceptable salts of the compounds of the invention. Also encompassed are salts which are themselves unsuitable for pharmaceutical uses but can be used for example for isolating or purifying the compounds of the invention.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine. Solvates refers for the purposes of the invention to those forms of the compounds of the invention which form, in the solid or liquid state, a complex by coordination with solvent molecules. Hydrates are a specific form of solvates in which the coordination takes place with water. Hydrates are preferred solvates in the context of the present invention. The present invention additionally encompasses prodrugs of the compounds of the invention. The term “prodrugs” encompasses compounds which themselves may be biologically active or inactive, but are converted during their residence time in the body into compounds of the invention (for example by metabolism or hydrolysis).

In particular, for the compounds of the formula (I) in which
Z represents a group of the formula

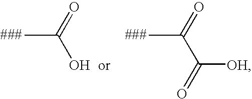

the present invention also includes hydrolyzable ester derivatives of these compounds. These are to be understood as meaning esters which can be hydrolyzed to the free carboxylic acids, as the compounds that are mainly active biologically, in physiological media, under the conditions of the biological tests described later and in particular in vivo by enzymatic or chemical routes. (C₁-C₄)-alkyl esters, in which the alkyl group can be straight—Chain or branched, are preferred as such esters. Particular preference is given to methyl, ethyl or tert-butyl esters (see also the corresponding definitions of the radical R⁸).

In the context of the present invention, the substituents have the following meaning, unless specified otherwise:

(C₁-C₆)-Alkyl, (C₁-C₅)-alkyl, (C₁-C₄)-alkyl and (C₁-C₃)-alkyl stand in the context of the invention for a straight—Chain or branched alkyl radical having respectively 1 to 6, 1 to 5, 1 to 4 and 1 to 3 carbon atoms. A straight—Chain or branched alkyl radical having 1 to 4, in particular 1 to 3, carbon atoms is preferred. Examples which may be preferably mentioned are: methyl, ethyl, n-propyl, isopropyl, n-butyl, isobutyl, sec-butyl, tert-butyl, 1-ethylpropyl, n-pentyl and n-hexyl.

(C₂-C₆)-Alkenyl, (C₂-C₅)-alkenyl and (C₂-C₄)-alkenyl stand in the context of the invention for a straight—Chain or branched alkenyl radical having respectively 2 to 6, 2 to 5 and 2 to 4 carbon atoms and 1 or 2 double bonds. A straight—Chain or branched alkenyl radical having 2 to 4 carbon atoms and one double bond is preferred. Examples which may be preferably mentioned are: vinyl, allyl, isopropenyl, n-but-2-en-1-yl, 2-methylprop-2-en-1-yl and n-but-3-en-1-yl.

(C₂-C₄)-Alkynyl stands in the context of the invention for a straight—Chain or branched alkynyl radical having 2 to 4 carbon atoms and one triple bond. A straight—Chain alkynyl radical having 2 to 4 carbon atoms is preferred. Examples which may be preferably mentioned are: ethynyl, n-prop-1-yn-1-yl, n-prop-2-yn-1-yl, n-but-2-yn-1-yl and n-but-3-yn-1-yl.

(C₁-C₄)-Alkanediyl and (C₁-C₃)-alkanediyl stand in the context of the invention for a straight—Chain or branched divalent alkyl radical having respectively 1 to 4 and 1 to 3 carbon atoms. In each case, a straight—Chain alkanediyl radical having respectively 1 to 4 and 1 to 3 carbon atoms is preferred. Examples which may be preferably mentioned are: methylene, ethane-1,2-diyl (1,2-ethylene), ethane-1,1-diyl, propane-1,3-diyl (1,3-propylene), propane-1,1-diyl, propane-1,2-diyl, propane-2,2-diyl, butane-1,4-diyl (1,4-butylene), butane-1,2-diyl, butane-1,3-diyl and butane-2,3-diyl.

(C₁-C₇)-Alkanediyl, (C₁-C₅)-alkanediyl and (C₃-C₇)-alkanediyl stand in the context of the invention for a straight—Chain or branched divalent alkyl radical having respectively 1 to 7, 1 to 5 and 3 to 7 carbon atoms. In each case, a straight—Chain alkanediyl radical having respectively 1 to 7, 1 to 5 and 3 to 7 carbon atoms is preferred. Examples which may be preferably mentioned are: methylene, ethane-1,2-diyl (1,2-ethylene), ethane-1,1-diyl, propane-1,3-diyl (1,3-propylene), propane-1,1-diyl, propane-1,2-diyl, propane-2,2-diyl, butane-1,4-diyl (1,4-butylene), butane-1,2-diyl, butane-1,3-diyl, butane-2,3-diyl, pentane-1,5-diyl (1,5-pentylene), pentane-2,4-diyl, 3-methyl-pentane-2,4-diyl and hexane-1,6-diyl (1,6-hexylene).

(C₂-C₄)-Alkenediyl and (C₂-C₃)-alkenediyl stand in the context of the invention for a straight—Chain or branched divalent alkenyl radical having respectively 2 to 4 and 2 to 3 carbon atoms and up to 2 double bonds. In each case, a straight—Chain alkenediyl radical having respectively 2 to 4 and 2 to 3 carbon atoms and one double bond is preferred. Examples which may be preferably mentioned are: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl and buta-1,3-diene-1,4-diyl.

(C₂-C₇)-Alkenediyl and (C₃-C₇)-alkenediyl stand in the context of the invention for a straight—Chain or branched divalent alkenyl radical having respectively 2 to 7 and 3 to 7 carbon atoms and up to 3 double bonds. In each case, a straight—Chain alkenediyl radical having respectively 2 to 7 and 3 to 7 carbon atoms and one double bond is preferred. Examples which may be preferably mentioned are: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl, buta-1,3-diene-1,4-diyl, pent-2-ene-1,5-diyl, hex-3-ene-1,6-diyl and hexa-2,4-diene-1,6-diyl.

(C₁-C₆)-Alkoxy and (C₁-C₄)-alkoxy stand in the context of the invention for a straight—Chain or branched alkoxy radical having respectively 1 to 6 and 1 to 4 carbon atoms. A straight—Chain or branched alkoxy radical having 1 to 4 carbon atoms is preferred.

Examples which may be preferably mentioned are: methoxy, ethoxy, n-propoxy, isopropoxy, n-butoxy, tert-butoxy, n-pentoxy and n-hexoxy.

(C₂-C₆)-Alkenyloxy stands in the context of the invention for a straight—Chain or branched alkenyloxy radical having 2 to 6 carbon atoms and one double bond in the alkenyl group. A straight—Chain or branched alkenyloxy radical having 3 or 4 carbon atoms is preferred. Examples which may be preferably mentioned are: allyloxy, (n-but-2-en-1-yl)oxy, (2-methylprop-2-en-1-yl)oxy and (n-but-3-en-1-yl)oxy.

(C₁-C₆)-Alkylthio and (C₁-C₄)-alkylthio stand in the context of the invention for a straight—Chain or branched alkylthio radical having respectively 1 to 6 and 1 to 4 carbon atoms. A straight—Chain or branched alkylthio radical having 1 to 4 carbon atoms is preferred. Examples which may be preferably mentioned are: methylthio, ethylthio, n-propylthio, isopropylthio, n-butylthio, tert-butylthio, n-pentylthio and n-hexylthio. (C₁-C₆)-Acyl [(C₁-C₆)-alkanoyl], (C₁-C₅)-acyl [(C₁-C₅)-alkanonoyl]and (C₁-C₄)-acyl [(C₁-C₄)-alkanoyl] stand in the context of the invention for a straight—Chain or branched alkyl radical having respectively 1 to 6, 1 to 5 and 1 to 4 carbon atoms which carries a doubly attached oxygen atom in the 1-position and is attached via the 1-position. A straight—Chain or branched acyl radical having 1 to 4 carbon atoms is preferred. Examples which may be preferably mentioned are: formyl, acetyl, propionyl, n-butyryl, isobutyryl and pivaloyl.

Mono-(C₁-C₆)-alkylamino and mono-(C₁-C₄)-alkylamino stand in the context of the invention for an amino group having a straight—Chain or branched alkyl substituent which has respectively 1 to 6 and 1 to 4 carbon atoms. A straight—Chain or branched monoalkylamino radical having 1 to 4 carbon atoms is preferred. Examples which may be preferably mentioned are: methylamino, ethylamino, n-propylamino, isopropylamino and tert-butylamino.

Di-(C₁-C₆)-alkylamino and di-(C₁-C₄)-alkylamino stand in the context of the invention for an amino group having two identical or different straight—Chain or branched alkyl substituents having respectively 1 to 6 and 1 to 4 carbon atoms. Straight—Chain or branched dialkylamino radicals having in each case 1 to 4 carbon atoms are preferred. Examples which may be preferably mentioned are: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Mono-(C₂-C₆)-alkenylamino stands in the context of the invention for an amino group having one straight—Chain or branched alkenyl substituent having 2 to 6 carbon atoms and one double bond. A straight—Chain or branched monoalkenylamino radical having 3 or 4 carbon atoms is preferred. Examples which may be preferably mentioned are: allylamino, (n-but-2-en-1-yl)amino, (2-methylprop-2-en-1-yl)amino and (n-but-3-en-1-yl)amino.

(C₁-C₆)-Acylamino and (C₁-C₄)-acylamino stand in the context of the invention for an amino group having a straight—Chain or branched acyl substituent which has respectively 1 to 6 and 1 to 4 carbon atoms and is attached via the carbonyl group. An acylamino radical having 1 to 4 carbon atoms is preferred. Examples which may be preferably mentioned are: formamido, acetamido, propionamido, n-butyramido and pivaloylamido.

(C₃-C₇)-Cycloalkyl, (C₃-C₆)-Cycloalkyl and (C₄-C₆)-Cycloalkyl stand in the context of the invention for a monocyclic saturated cycloalkyl group having respectively 3 to 7, 3 to 6 and 4 to 6 carbon atoms. A cycloalkyl radical having 3 to 6 carbon atoms is preferred. Examples which may be preferably mentioned are: cyclopropyl, cyclobutyl, cyclopentyl, cyclohexyl and cycloheptyl.

(C₄-C₇)-Cycloalkenyl, (C₄-C₆)-Cycloalkenyl and (C₅-C₆)-Cycloalkenyl stand in the context of the invention for a monocyclic cycloalkyl group having respectively 4 to 7, 4 to 6 and 5 or 6 carbon atoms and one double bond. A cycloalkenyl radical having 4 to 6, particularly preferably 5 or 6, carbon atoms is preferred. Examples which may be preferably mentioned are: cyclobutenyl, cyclopentenyl, cyclohexenyl and cycloheptenyl. 5- to 7-membered heterocyclyl stands in the context of the invention for a saturated or partially unsaturated heterocycle having 5 to 7 ring atoms which contains one or two ring heteroatoms from the group consisting of N and O and is attached via ring carbon atoms and/or, if appropriate, ring nitrogen atoms. 5- or 6-membered saturated heterocyclyl having one or two ring heteroatoms from the group consisting of N and O is preferred.

Examples which may be mentioned are: pyrrolidinyl, pyrrolinyl, pyrazolidinyl, tetrahydrofuranyl, piperidinyl, piperazinyl, dihydropyranyl, tetrahydropyranyl, morpholinyl, hexahydroazepinyl and hexahydro-1,4-diazepinyl. Preference is given to pyrrolidinyl, tetrahydrofuranyl, piperidinyl, piperazinyl, tetrahydropyranyl and morpholinyl. 5- or 6-membered heteroaryl stands in the context of the invention for an aromatic heterocycle (heteroaromatic) having a total of 5 or 6 ring atoms which contains one or two ring heteroatoms from the group consisting of N, O and S and is attached via ring carbon atoms and/or, if appropriate, a ring nitrogen atom. Examples which may be mentioned are: furyl, pyrrolyl, thienyl, pyrazolyl, imidazolyl, thiazolyl, oxazolyl, isoxazolyl, isothiazolyl, pyridyl, pyrimidinyl, pyridazinyl and pyrazinyl. Preference is given to thienyl, pyridyl, pyrimidinyl, pyridazinyl and pyrazinyl.

Halogen includes in the context of the invention fluorine, chlorine, bromine and iodine. Preference is given to chlorine or fluorine.

If radicals in the compounds according to the invention are substituted, the radicals, unless specified otherwise, may be mono- or polysubstituted. In the context of the present invention, for all radicals that occur more than once, their meanings are independent of one another. Substitution by one, two or three identical or different substituents is preferred. Particular preference is given to substitution by one or two identical or different substituents, and very particular preference is given to substitution by one substituent.

In the context of the present invention, preference is given to compounds of the formula (I) in which
R¹ is (C₁-C₄)-alkyl or a group of the formula —C(═O)—R^{1A} in which
R^{1A} represents (C₁-C₄)-alkyl, hydroxyl, (C₁-C₄)-alkoxy, allyloxy, mono-(C₁-C₄-alkylamino or allylamino,
R² is hydrogen, methyl or ethyl,
R³ is a substituent selected from the group consisting of fluorine, chlorine, cyano, methyl, ethyl, methoxy, ethoxy, trifluoromethyl and trifluoromethoxy,
m is the number 0, 1 or 2,
where, if two substituents R³ are present, their meanings may be identical or different,
A is O or NH,
M is a group of the formula

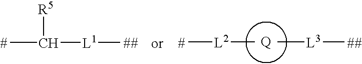

where
# represents the point of attachment to group A and
## represents the point of attachment to group Z,
R⁵ represents hydrogen, methyl or ethyl,
L¹ represents (C₃-C₇)-alkanediyl, (C₃-C₇)-alkenediyl or a group of the formula
*-L^{1A}-V-L^{1B}** in which
* denotes the point of attachment to the group —CHR⁵,
** denotes the point of attachment to group Z,

L^{A} denotes (C₁-C₃)-alkanediyl which may be mono- or disubstituted by methyl,
L^{1B} denotes (C₁-C₃)-alkanediyl and
V denotes O or N—CH₃,
L² represents a bond, methylene, ethane-1,1-diyl or ethane-1,2-diyl,
L³ represents (C₁-C₃)-alkanediyl or a group of the formula •—W—CH₂—•• or
•—W—CH₂—CH₂—•• in which
• denotes the point of attachment to ring Q,
•• denotes the point of attachment to group Z and
W denotes O or N—R⁷ in which
R⁷ represents hydrogen or (C₁-C₃)-alkyl, and
Q represents cyclobutyl, cyclopentyl, cyclopentenyl, cyclohexyl, pyrrolidinyl, piperidinyl, tetrahydrofuranyl, tetrahydropyranyl, morpholinyl or phenyl, each of which may be substituted up to two times by identical or different radicals selected from the group consisting of fluorine, methyl, ethyl, trifluoromethyl, hydroxyl, methoxy and ethoxy, and
Z is a group of the formula

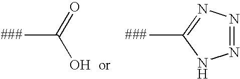

in which
### represents the point of attachment to group L¹ or L³ and their salts, solvates and solvates of the salts.

In the context of the present invention, particular preference is given to compounds of the formula (I) in which
R¹ represents ethyl, n-propyl or a group of the formula —C(═O)—R^{1A} in which
R^{1A} represents ethyl, n-propyl, ethoxy, allyloxy, ethylamino, n-propylamino or allylamino,
R² is hydrogen or methyl,
R³ is fluorine, chlorine or methyl,
m is the number 0 or 1,
A is O or NH,
M is the group of the formula

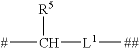

in which
# represents the point of attachment to group A and
## represents the point of attachment to group Z,
R⁵ represents hydrogen or methyl, and
L¹ represents butane-1,4-diyl, pentane-1,5-diyl or a group of the formula *-L^{1A}-O-L^{1B}-** in which
* denotes the point of attachment to the group —CHR⁵,
** denotes the point of attachment to group Z,
L^{1A} denotes methylene or ethane-1,2-diyl which may be mono- or disubstituted by methyl, and
L^{1B} denotes methylene or ethane-1,2-diyl, and
Z represents the group of the formula

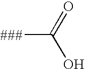

in which
### represents the point of attachment to group L¹,
and their salts, solvates and solvates of the salts.

The individual definitions of radicals given in the respective combinations and preferred combinations of radicals are, independently of the given combination of radicals in question, also optionally replaced by radical definitions of other combinations.

Particular preference is given to combinations of two or more of the preferred ranges mentioned above.

In the context of the present invention, very particular preference is given to the compounds mentioned below:

(6R)-6-({5-[(1E)-3-ethoxy-2-methyl-3-oxoprop-1-en-1-yl]-6-phenylfuro[2,3-d]pyrimidin-4-yl}oxy)heptanoic acid,

(6R)-6-({5-[(1E)-3-(ethylamino)-2-methyl-3-oxoprop-1-en-1-yl]-6-phenylfuro[2,3-d]pyrimidin-4-yl}oxy)heptanoic acid and

(6R)-6-({5-[(1E)-2-methyl-3-oxo-3-(propylamino)prop-1-en-1-yl]-6-phenylfuro[2,3-d]pyrimidin-4-yl}oxy)heptanoic acid

and their salts, solvates and solvates of the salts.

The invention furthermore provides a process for preparing the compounds of the formula (I) according to the invention in which Z represents —COOH or —C(═O)—COOH, characterized in that a compound of the formula (II)

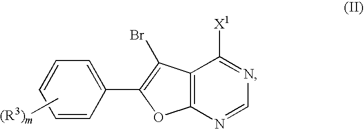

in which R³ and m have the meanings given above and
X¹ is a leaving group, such as, for example, halogen, in particular chlorine, is reacted in an inert solvent in the presence of a base with a compound of the formula (III)

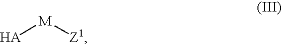

in which A and M have the meanings given above and
Z¹ is cyano or a group of the formula —[(O)]_{y}—COOR^{8A} in which
y represents the number 0 or 1 and
R^{8A} represents (C₁-C₄)-alkyl,
to give a compound of the formula (IV)

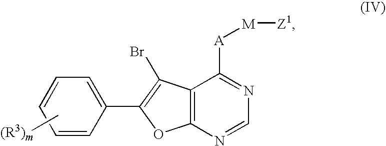

in which A, M, Z¹, R³ and m each have the meanings given above,
which is then either
[A] coupled in an inert solvent in the presence of a base and a suitable palladium catalyst with a boronic acid derivative of the formula (V) or an olefin of the formula (VI)

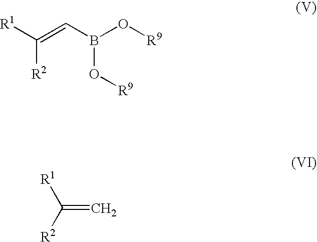

in which R¹ and R² have the meanings given above and
R⁹ is hydrogen or (C₁-C₄)-alkyl or both radicals R⁹ together form a —CH₂—CH₂—, —C(CH₃)₂—C(CH₃)₂— or —CH₂—C(CH₃)₂—CH₂— bridge,
to give a compound of the formula (VII)

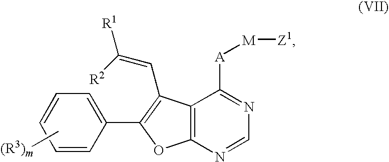

in which A, M, Z¹, R¹, R², R³ and m each have the meanings given above, or
[B] initially converted in an inert solvent in the presence of a base and a suitable palladium catalyst with a vinylboronic acid derivative of the formula (VIII)

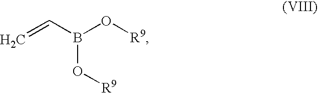

in which R⁹ has the meaning given above
into a compound of the formula (IX)

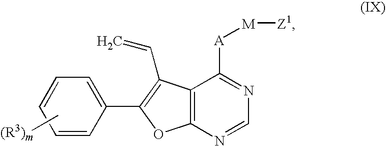

in which A, M, Z¹, R³ and m each have the meanings given above,
then oxidized by reaction with ozone and subsequent treatment with a sulfide to give a compound of the formula (X)

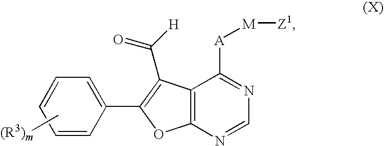

in which A, M, Z¹, R³ and m each have the meanings given above,
and then coupled in an inert solvent in the presence of a base with a phosphorus ylide of the formula (XI) or a phosphonate of the formula (XII)

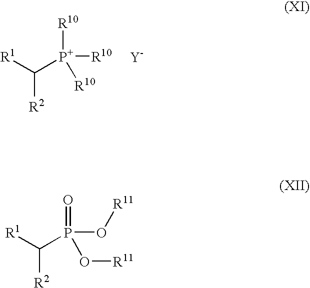

in which R¹ and R² have the meanings given above and R¹⁰ represents phenyl or o-, m- or p-tolyl,
R¹¹ represents (C₁-C₄)-alkyl and
Y⁻ represents a halide anion,
to give a compound of the formula (VII)

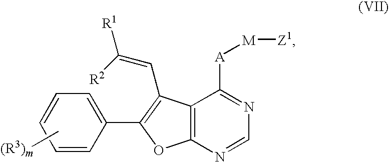

in which A, M, Z¹, R¹, R², R³ and m each have the meanings given above,
and the compounds of the formula (VII) are finally converted by hydrolysis of the ester or cyano group Z¹ into the carboxylic acids of the formula (I-A)

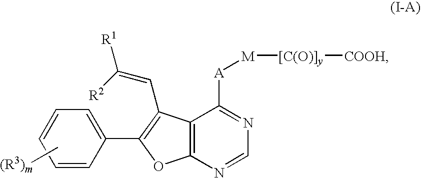

in which A, M, R¹, R², R³, m and y each have the meanings given above, and these are, if appropriate, reacted with the appropriate (i) solvents and/or (ii) bases or acids to give their solvates, salts and/or solvates of the salts.

Inert solvents for the process step (II)+(III)→(IV) are, for example, ethers, such as diethyl ether, methyl tert-butyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethane, tetrachloroethane, trichloroethylene, chlorobenzene or chlorotoluene, or other solvents, such as dimethylformamide (DMF), dimethyl sulfoxide (DMSO), N,N′-dimethyl-propyleneurea (DMPU), N-methylpyrrolidone (NMP) or acetonitrile. It is also possible to use mixtures of the solvents mentioned. Preference is given to using tetrahydrofuran, toluene, dimethylformamide, dimethyl sulfoxide or mixtures of these solvents.

However, if appropriate, the process step (II)+(III)→(IV) can also be carried out in the absence of a solvent.

Suitable bases for the process step (II)+(III)→(IV) are customary inorganic or organic bases. These preferably include alkali metal hydroxides, such as, for example, lithium hydroxide, sodium hydroxide or potassium hydroxide, alkali metal or alkaline earth metal carbonates, such as lithium carbonate, sodium carbonate, potassium carbonate, calcium carbonate or cesium carbonate, alkali metal alkoxides, such as sodium tert-butoxide or potassium tert-butoxide, alkali metal hydrides, such as sodium hydride or potassium hydride, amides, such as lithium bis(trimethylsilyl)amide or potassium bis(trimethylsilyl)amide or lithium diisopropylamide, organometallic compounds, such as butyllithium or phenyllithium, or organic amines, such as triethylamine, N-methyl-morpholine, N-methylpiperidine, N,N-diisopropylethylamine or pyridine.

In the case of the reaction with alcohol derivatives [A in (III)=O], phosphazene bases (so-Called “Schwesinger bases”), such as, for example, P2-t-Bu or P4-t-Bu are likewise expedient [cf., for example, R. Schwesinger, H. Schlemper, Angew. Chem. Int. Ed. Engl. 26, 1167 (1987); T. Pietzonka, D. Seebach, Chem. Ber. 124, 1837 (1991)].

In the reaction with amine derivatives [A in (III)=N], the base used is preferably a tertiary amine, such as, in particular, N,N-diisopropylethylamine, sodium tert-butoxide or sodium hydride. However, if appropriate, these reactions can—if an excess of the amine component (III) is used—also be carried out without the addition of an auxiliary base. In the reaction with alcohol derivatives [A in (III)=O], preference is given to sodium hydride, potassium carbonate or cesium carbonate or the phosphazene bases P2-t-Bu and P4-t-Bu.

If appropriate, the process step (II)+(III)→(IV) can advantageously be carried out with addition of a crown ether.

In a further process variant, the reaction (II)+(III)→(IV) can also be carried out in a two-phase mixture consisting of an aqueous alkali metal hydroxide solution as base and one of the hydrocarbons or halogenated hydrocarbons mentioned above as further solvent, using a phase transfer catalyst such as tetrabutylammonium hydrogensulfate or tetrabutylammonium bromide.

The process step (II)+(III)→(IV) is, in the reaction with amine derivatives [A in (III)=N], generally carried out in a temperature range of from −20° C. to +150° C., preferably at from 0° C. to +100° C. In the reaction with alcohol derivatives [A in (III)=O], the reaction is generally carried out in a temperature range of from −20° C. to +120° C., preferably at from −10° C. to +80° C.

Inert solvents for the process steps (IV)+(V)→(VII) and (IV)+(VIII)→(IX) are, for example, alcohols, such as methanol, ethanol, n-propanol, isopropanol, n-butanol or tert-butanol, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide, dimethyl sulfoxide, N,N′-dimethylpropyleneurea, N-methylpyrrolidone, pyridine, acetonitrile or else water. It is also possible to use mixtures of the solvents mentioned. Preference is given to a mixture of tetrahydrofuran and water. Suitable bases for the process steps (IV)+(V)→(VII) and (IV)+(VIII)→(IX) are customary inorganic bases. These include in particular alkali metal hydroxides, such as, for example, lithium hydroxide, sodium hydroxide or potassium hydroxide, alkali metal bicarbonates, such as sodium bicarbonate or potassium bicarbonate, alkali metal carbonates or alkaline earth metal carbonates, such as lithium carbonate, sodium carbonate, potassium carbonate, calcium carbonate or cesium carbonate, or alkali metal hydrogenphosphates, such as disodium hydrogenphosphate or dipotassium hydrogenphosphate. Preference is given to using sodium carbonate or potassium carbonate.

The reactions (IV)+(V)→(VII) and (IV)+(VIII)→(IX) are generally carried out in a temperature range of from +20° C. to +150° C., preferably at from +50° C. to +100° C. Inert solvents for the process step (IV)+(VI)→(VII) are, for example, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide, dimethyl sulfoxide, N,N′-dimethylpropyleneurea, N-methylpyrrolidone, pyridine or acetonitrile. It is also possible to use mixtures of the solvents mentioned. Preference is given to using dimethylformamide.

The process step (IV)+(VI)→(VII) is customarily carried out in the presence of a tertiary amine base. Suitable for this purpose are in particular amines such as triethylamine, tri-n-butylamine, N,N-diisopropylethylamine, N-methylpiperidine or N-methylmorpholine. Preference is given to using triethylamine or N,N-diisopropylethylamine.

The addition of tetraalkylammonium salts, such as, for example, tetra-n-butylammonium bromide, may, if appropriate, be advantageous in the reaction (IV)+(VI)→(VII).

The reaction (IV)+(VI)→(VII) is generally carried out in a temperature range of from +50° C. to +200° C., preferably at from +80° C. to +150° C.

The process steps (IV)+(V)→(VII) and (IV)+(VIII)→(IX) [“Suzuki coupling”] and (IV)+(VI)→(VII) [“Heck reaction”] are in each case carried out in the presence of a palladium catalyst. Suitable for this purpose are palladium compounds customary for such coupling reactions, such as, for example, palladium(II) acetate, tetrakis(triphenyl-phosphine)palladium(0), bis(triphenylphosphine)palladium(II) chloride, bis(tri-o-tolylphosphine)palladium(II) chloride, bis(acetonitrile)palladium(II) chloride and [1,1′-bis(diphenylphosphino)ferrocene]dichloropalladium(II)/dichloromethane complex [cf., for example, J. Hassan et al., Chem. Rev. 102, 1359-1469 (2002)]. Preference is given to using bis(triphenylphosphine)palladium(II) chloride or bis(tri-o-tolylphosphine)palladium(II) chloride.

The ozonolysis in the process step (IX)→(X) is carried out according to known methods using an ozone generator, preferably in alcohol/dichloromethane mixtures as solvent in a temperature range of from −100° C. to −60° C. For the reductive aftertreatment of the reaction mixture, preference is given to using sulfides, such as, for example, dimethyl sulfide.

Inert solvents for the process step (X)+(XI) or (XII)→(VII) are, for example, ethers, such as diethyl ether, tert-butyl methyl ether, dioxane, tetrahydrofuran, glycol dimethyl ether or diethylene glycol dimethyl ether, hydrocarbons, such as benzene, toluene, xylene, pentane, hexane, cyclohexane or mineral oil fractions, or other solvents, such as dimethylformamide, dimethyl sulfoxide, N,N′-dimethylpropyleneurea or N-methylpyrrolidone. It is also possible to use mixtures of the solvents mentioned. Preference is given to using tetrahydrofuran.

Suitable bases for the process step (X)+(XI) or (XII)→(VII) are bases customary for Wittig or Wittig-Horner reactions of this type. These include in particular alkali metal hydrides, such as sodium hydride or potassium hydride, alkali metal alkoxides, such as sodium tert-butoxide or potassium tert-butoxide, amides, such as lithium bis(trimethylsilyl)amide or potassium bis(trimethylsilyl)amide or lithium diisopropylamide, or organometallic compounds, such as butyllithium or phenyllithium. Preference is given to sodium hydride.

The reactions (X)+(XI) and (XII)→(VII) are generally carried out in a temperature range of from −20° C. to +60° C., preferably at from 0° C. to +40° C.

The hydrolysis of the ester or nitrile group Z¹ in the process step (VII)→(I-A) is carried out by customary methods by treating the esters or nitriles in inert solvents with acids or bases, where in the latter case the salts initially formed are converted by treatment with acid into the free carboxylic acids. In the case of the tert-butyl esters, the ester cleavage is preferably carried out using acids.

Suitable inert solvents for these reactions are water or the organic solvents customary for ester cleavage. These preferably include alcohols, such as methanol, ethanol, n-propanol, isopropanol, n-butanol or tert-butanol, or ethers, such as diethyl ether, tetrahydrofuran, dioxane or glycol dimethyl ether, or other solvents, such as acetone, dichloromethane, dimethylformamide or dimethyl sulfoxide. It is also possible to use mixtures of the solvents mentioned. In the case of a basic ester hydrolysis, preference is given to using mixtures of water with dioxane, tetrahydrofuran, methanol and/or ethanol, and for nitrile hydrolysis, preference is given to using water and/or n-propanol. In the case of the reaction with trifluoroacetic acid, preference is given to using dichloromethane, and in the case of the reaction with hydrogen chloride, preference is given to using tetrahydrofuran, diethyl ether, dioxane or water.

Suitable bases are the customary inorganic bases. These preferably include alkali metal hydroxides or alkaline earth metal hydroxides, such as, for example, sodium hydroxide, lithium hydroxide, potassium hydroxide or barium hydroxide, or alkali metal carbonates or alkaline earth metal carbonates, such as sodium carbonate, potassium carbonate or calcium carbonate. Particular preference is given to sodium hydroxide or lithium hydroxide.

Acids suitable for the ester cleavage are, in general, sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, if appropriate with addition of water. Preference is given to hydrogen chloride or trifluoroacetic acid in the case of the tert-butyl esters and to hydrochloric acid in the case of the methyl esters.

The ester cleavage is generally carried out in a temperature range of from 0° C. to +100° C., preferably at from +0° C. to +50° C. The nitrile hydrolysis is generally carried out in a temperature range of from +50° C. to +150° C., preferably at from +80° C. to +120° C. The reactions mentioned can be carried out at atmospheric, elevated or reduced pressure (for example from 0.5 to 5 bar). In general, the reactions are carried out at atmospheric pressure.

The compounds of the formula (I) according to the invention in which Z represents a group of the formula

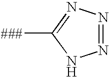

can be prepared by reacting compounds of the formula (VII) in which Z¹ represents cyano in an inert solvent with an alkali metal azide in the presence of ammonium chloride or with trimethylsilyl azide, if appropriate in the presence of a catalyst. Inert solvents for this reaction are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, or other solvents, such as dimethyl sulfoxide, dimethylformamide, N,N′-dimethylpropyleneurea or N-methylpyrrolidone. It is also possible to use mixtures of the solvents mentioned.

Preference is given to using toluene.

A suitable azide reagent is in particular sodium azide in the presence of ammonium chloride or trimethylsilyl azide. The latter reaction can advantageously be carried out in the presence of a catalyst. Suitable for this purpose are in particular compounds such as di-n-butyltin oxide, trimethylaluminum or zinc bromide. Preference is given to using trimethylsilyl azide in combination with di-n-butyltin oxide.

The reaction is generally carried out in a temperature range of from +50° C. to +150° C., preferably at from +60° C. to +110° C. The reaction can be carried out at atmospheric, elevated or reduced pressure (for example from 0.5 to 5 bar). In general, the reaction is carried out at atmospheric pressure.

The compounds of the formula (I) according to the invention in which Z represents a group of the formula

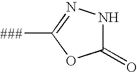

can be prepared by converting compounds of the formula (VII) in which Z¹ represents methoxy- or ethoxycarbonyl [y=0] initially in an inert solvent with hydrazine into compounds of the formula (XIII)

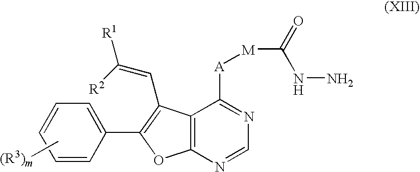

in which A, M, R¹, R², R³ and m each have the meanings given above, and then reacting in an inert solvent with phosgene or a phosgene equivalent, such as, for example, N,N′-Carbonyl diimidazole.

Suitable inert solvents for the first step of this reaction sequence are in particular alcohols, such as methanol, ethanol, n-propanol, isopropanol, n-butanol or tert-butanol, or ethers, such as diethyl ether, dioxane, tetrahydrofuran, glycol dimethyl ether or diethylene glycol dimethyl ether. It is also possible to use mixtures of these solvents. Preference is given to using a mixture of methanol and tetrahydrofuran. The second reaction step is preferably carried out in an ether, in particular in tetrahydrofuran. The reactions are generally carried out in a temperature range of from 0° C. to +70° C., under atmospheric pressure.

The compounds of the formula (I) according to the invention in which L¹ represents a group of the formula *L^{1A}-V-L^{1B}-**, where L^{1A}, L^{1B} and V have the meanings given above, can alternatively also be prepared by initially reacting compounds of the formula (II)

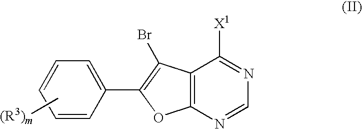

in which X¹, R³ and m have the meanings given above in an inert solvent in the presence of a base with a compound of the formula (XIV)

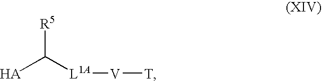

in which A, L^{1A}, V and R⁵ each have the meanings given above and
T is hydrogen or a temporary O- or N-protective group,
to give compounds of the formula (XV)

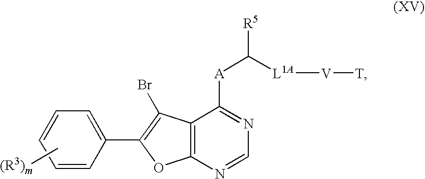

in which A, L^{1A}, T, V, R³, R⁵ and m each have the meanings given above, then—after removal of any protective group T present—converting these in an inert solvent in the presence of a base with a compound of the formula (XVI)

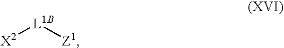

in which L^{1B} and Z¹ have the meanings given above and
X² is a leaving group, such as, for example, halogen, mesylate, tosylate or triflate, or, if L^{1B} is —CH₂CH₂—, with a compound of the formula (XVII)

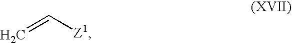

in which Z¹ has the meaning given above,
into compounds of the formula (IV-A)

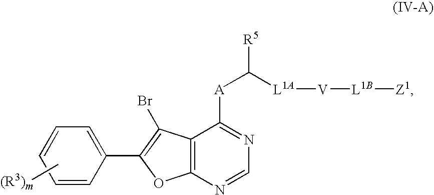

in which A, L^{1A}, L^{1B}, V, Z¹, R³, R⁵ and m each have the meanings given above, and subsequently reacting further according to the process described above (see also reaction scheme 2 below).

In an analogous manner, the compounds of the formula (I) according to the invention in which L³ is a group of the formula φ—W—CH₂—•• or •—W—CH₂—CH₂—•• in which W has the meaning given above can also be prepared by initially reacting compounds of the formula (II)

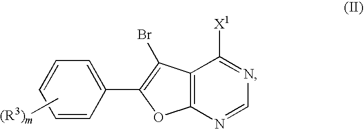

in which X¹, R³ and m have the meanings given above,
in an inert solvent in the presence of a base with a compound of the formula (XVIII)

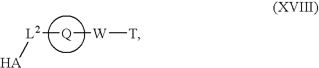

in which A, L², Q and W each have the meanings given above and
T is hydrogen or a temporary O- or N-protective group,
to give compounds of the formula (XIX)

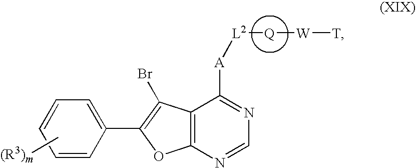

in which A, L², Q, T, W, R³ and m each have the meanings given above, then—after removal of any protective group T present—converting these in an inert solvent in the presence of a base with a compound of the formula (XX)

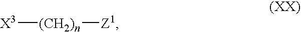

in which Z¹ has the meaning given above,
n is the number 1 or 2 and
X³ is a leaving group, such as, for example, halogen, mesylate, tosylate or triflate, or, if L³ is •—W—CH₂CH₂—••, with a compound of the formula (XVII)

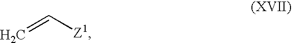

in which Z¹ has the meaning given above,
into compounds of the formula (IV-B)

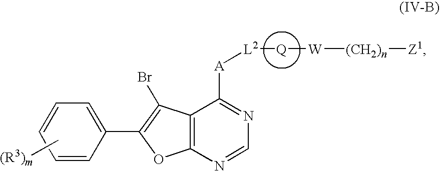

in which A, L², Q, W, Z¹, R³, m and n each have the meanings given above, and subsequently reacting further according to the process described above (see also reaction scheme 2 below).

For the process steps (II)+(XIV)→(XV), (XV)+(XVI) or (XVII)→(IV-A), (II)+(XVIII)→(XIX) and (XIX)+(XX) or (XVII)→(IV-B), the reaction parameters described above for the reaction (II)+(III)→(IV), such as solvents, bases and reaction temperatures, are used in an analogous manner.

For their part, the compounds of the formula (II) can be prepared, for example, by initially converting phenacyl bromides of the formula (XXI)

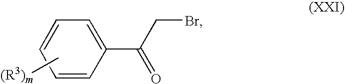

in which R³ and m have the meanings mentioned above,
with malononitrile in the presence of a base, such as, for example, diethylamine, into 2-aminofuronitriles of the formula (XXII)

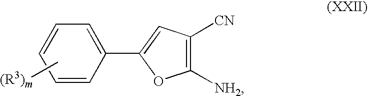

in which R³ and m have the meanings mentioned above,
then condensing these with formamide to give 4-aminofuropyrimidines of the formula (XXIII)

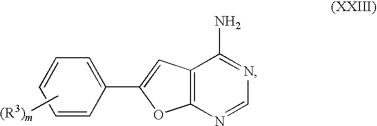

in which R³ and m have the meanings mentioned above,
then brominating with N-bromosuccinimide to give compounds of the formula (XXIV)

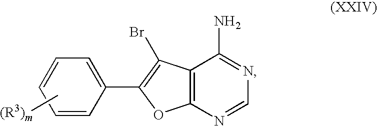

in which R³ and m have the meanings mentioned above,
and subsequently reacting with isoamyl nitrite in the presence of a chloride source such as hydrogen chloride or copper(II) chloride to give compounds of the formula (II-A)

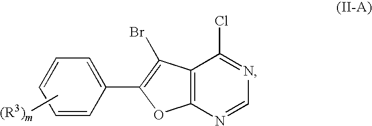

in which R³ and m have the meanings mentioned above (see also reaction scheme 3 below).

The compounds of the formulae (III), (V), (VI), (VIII), (XI), (XII), (XIV), (XVI), (XVII), (XVIII), (XX) and (XXI) are commercially available, known from the literature or can be prepared analogously to processes known from the literature.

The preparation of the compounds according to the invention can be illustrated in an exemplary manner by the synthesis schemes below:

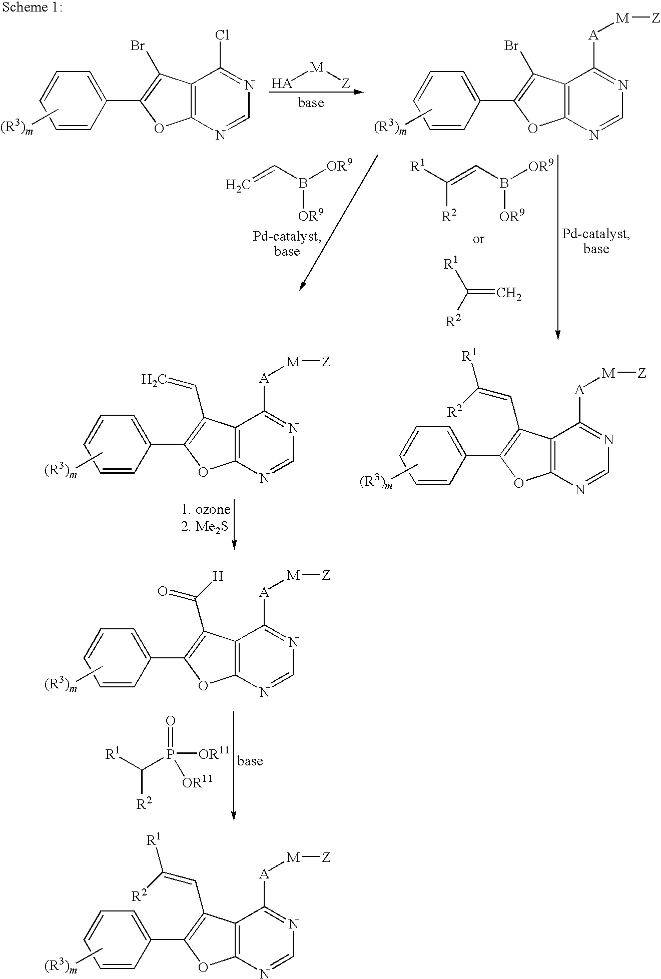

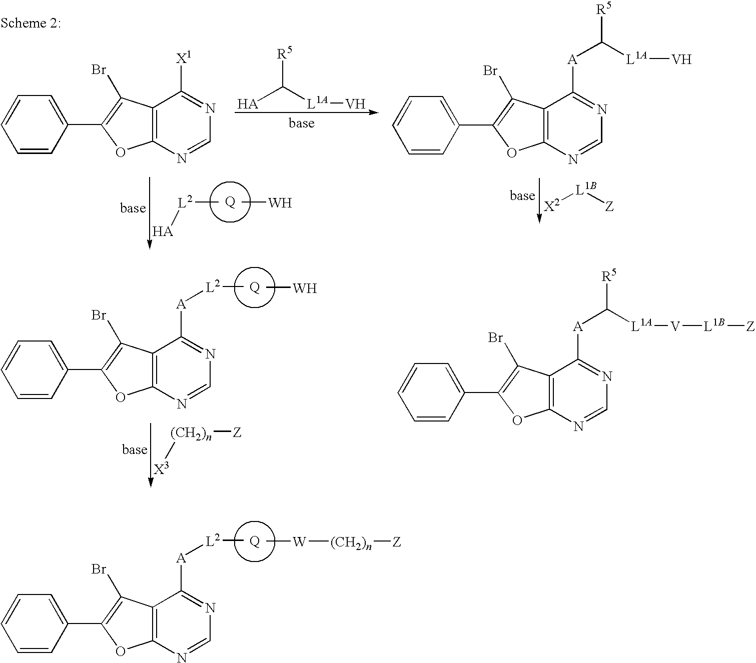

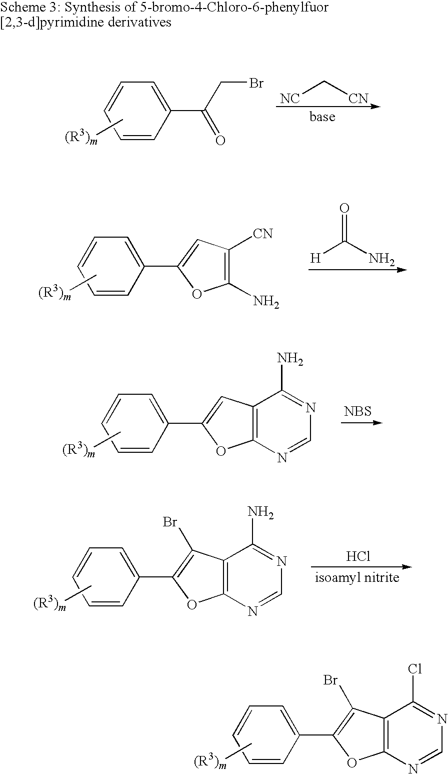

The compounds according to the invention possess valuable pharmacological properties and can be used for the prevention and treatment of diseases in humans and animals. The compounds according to the invention are chemically and metabolically stable, non-prostanoid activators of the IP receptor which mimic the biological action of PGI₂.

They are thus suitable in particular for the prophylaxis and/or treatment of cardiovascular diseases such as stable and unstable angina pectoris, of hypertension and heart failure, pulmonary hypertension, for the prophylaxis and/or treatment of thromboembolic diseases and ischaemias such as myocardial infarction, stroke, transient and ischemic attacks and subarachnoid haemorrhage, and for the prevention of restenoses such as after thrombolytic treatments, percutaneous transluminal angioplasty (PTA), coronary angioplasty (PTCA) and bypass surgery.

The compounds according to the invention are particularly suitable for the treatment and/or prophylaxis of pulmonary hypertension (PH) including its various manifestations. The compounds of the invention are therefore particularly suitable for the treatment and/or prophylaxis of pulmonary arterial hypertension (PAH) and its subtypes such as idiopathic and familial pulmonary arterial hypertension, and the pulmonary arterial hypertension which is associated for example with portal hypertension, fibrotic disorders, HIV infection or inappropriate medications or toxins.

The compounds of the invention can also be used for the treatment and/or prophylaxis of other types of pulmonary hypertension. Thus, for example, they can be employed for the treatment and/or prophylaxis of pulmonary hypertension associated with left atrial or left ventricular disorders and with left heart valve disorders. In addition, the compounds of the invention are suitable for the treatment and/or prophylaxis of pulmonary hypertension associated with chronic obstructive pulmonary disease, interstitial pulmonary disease, pulmonary fibrosis, sleep apnoea syndrome, disorders with alveolar hypoventilation, altitude sickness and pulmonary development impairments.

The compounds of the invention are furthermore suitable for the treatment and/or prophylaxis of pulmonary hypertension based on chronic thrombotic and/or embolic disorders such as, for example, thromboembolism of the proximal pulmonary arteries, obstruction of the distal pulmonary arteries and pulmonary embolism. The compounds of the invention can further be used for the treatment and/or prophylaxis of pulmonary hypertension connected with sarcoidosis, histiocytosis X or lymphangioleiomyomatosis, and where the pulmonary hypertension is caused by external compression of vessels (lymph nodes, tumor, fibrosing mediastinitis).

In addition, the compounds according to the invention can also be used for the treatment and/or prophylaxis of peripheral and cardial vascular diseases, peripheral occlusive diseases (PAOD, PVD) and disturbances of peripheral blood flow.

Furthermore, the compounds according to the invention can be used for the treatment of arteriosclerosis, hepatitis, asthmatic diseases, chronic obstructive pulmonary diseases (COPD), pulmonary edema, fibrosing lung diseases such as idiopathic pulmonary fibrosis (IPF) and ARDS, inflammatory vascular diseases such as scleroderma and lupus erythematosus, renal failure, arthritis and osteoporosis, and also for the prophylaxis and/or treatment of cancers, especially of metastasizing tumors.

Moreover, the compounds according to the invention can also be used as an addition to the preserving medium of an organ transplant, e.g. kidneys, lungs, heart or islet cells. The present invention further relates to the use of the compounds according to the invention for the treatment and/or prevention of diseases, especially of the aforementioned diseases.

The present invention further relates to the use of the compounds according to the invention for the production of a medicament for the treatment and/or prevention of diseases, especially of the aforementioned diseases.

The present invention further relates to a method for the treatment and/or prevention of diseases, especially of the aforementioned diseases, using an effective amount of at least one of the compounds according to the invention.

The compounds of the invention can be employed alone or, if required, in combination with other active ingredients. The present invention further relates to medicaments comprising at least one of the compounds of the invention and one or more further active ingredients, especially for the treatment and/or prevention of the aforementioned disorders. Suitable active ingredients for combinations are by way of example and preferably:

organic nitrates and NO donors such as, for example, sodium nitroprusside, nitroglycerin, isosorbide mononitrate, isosorbide dinitrate, molsidomine or SIN-1, and inhaled NO;

compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil;

NO-independent but heme-dependent stimulators of guanylate cyclase such as in particular the compounds described in WO 00/06568, WO 00/06569, WO 02/42301 and WO 03/095451;

NO- and heme-independent activators of guanylate cyclase, such as in particular the compounds described in WO 01/19355, WO 01/19776, WO 01/19778, WO 01/19780, WO 02/070462 and WO 02/070510;

compounds which inhibit human neutrophile elastase (HNE), such as, for example, sivelestat, DX-890 (Reltran), elafin or in particular the compounds described in WO 03/053930, WO 2004/020410, WO 2004/020412, WO 2004/024700, WO 2004/024701, WO 2005/080372, WO 2005/082863 and WO 2005/082864;

compounds which inhibit the signal transduction cascade, for example and preferably from the group of kinase inhibitors, in particular from the group of tyrosine kinase and/or serine/threonine kinase inhibitors;

compounds which inhibit soluble epoxide hydrolase (sEH), such as, for example, N,N′-dicyclohexylurea, 12-(3-adamantan-1-yl-ureido)dodecanoic acid or 1-adamantan-1-yl-3-{5-[2-(2-ethoxyethoxy)ethoxy]pentyl}urea;

compounds which influence the energy metabolism of the heart, such as by way of example and preferably etomoxir, dichloroacetate, ranolazine or trimetazidine;

agonists of VPAC receptors, such as by way of example and preferably the vasoactive intestinal polypeptide (VIP);

agents having an antithrombotic effect, for example and preferably from the group of platelet aggregation inhibitors, of anticoagulants or of profibrinolytic substances; active ingredients which lower blood pressure, for example and preferably from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors and diuretics; and/or active ingredients which alter lipid metabolism, for example and preferably from the group of thyroid receptor agonists, cholesterol synthesis inhibitors such as by way of example and preferably HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists. In a preferred embodiment of the invention, the compounds of the invention are administered in combination with a kinase inhibitor such as by way of example and preferably canertinib, imatinib, gefitinib, erlotinib, lapatinib, lestaurtinib, lonafarnib, pegaptinib, pelitinib, semaxanib, tandutinib, tipifarnib, vatalanib, sorafenib, sunitinib, bortezomib, lonidamine, leflunomide, fasudil, or Y-27632.

Agents having an antithrombotic effect preferably mean compounds from the group of platelet aggregation inhibitors, of anticoagulants or of profibrinolytic substances. In a preferred embodiment of the invention, the compounds of the invention are administered in combination with a platelet aggregation inhibitor such as by way of example and preferably aspirin, clopidogrel, ticlopidine or dipyridamole.

In a preferred embodiment of the invention, the compounds of the invention are administered in combination with a thrombin inhibitor such as by way of example and preferably ximelagatran, melagatran, bivalirudin or clexane.

In a preferred embodiment of the invention, the compounds of the invention are administered in combination with a GPIIb/IIIa antagonist such as by way of example and preferably tirofiban or abciximab.

In a preferred embodiment of the invention, the compounds of the invention are administered in combination with a factor Xa inhibitor such as by way of example and preferably rivaroxaban, DU-176b, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or a low molecular weight (LMW) heparin derivative.

In a preferred embodiment of the invention, the compounds of the invention are administered in combination with a vitamin K antagonist such as by way of example and preferably coumarin.

Agents which lower blood pressure preferably mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors, and diuretics.

In a preferred embodiment of the invention, the compounds of the invention are administered in combination with a calcium antagonist such as by way of example and preferably nifedipine, amlodipine, verapamil or diltiazem.

In a preferred embodiment of the invention, the compounds of the invention are administered in combination with an alpha-1 receptor blocker such as by way of example and preferably prazosin.

In a preferred embodiment of the invention, the compounds of the invention are administered in combination with a beta-receptor blocker such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin All antagonist such as by way of example and preferably losartan, candesartan, valsartan, telmisartan or embusartan. In a preferred embodiment of the invention, the compounds of the invention are administered in combination with an ACE inhibitor such as by way of example and preferably enalapril, captopril, lisinopril, ramipril, delapril, fosinopril, quinopril, perindopril or trandopril.

In a preferred embodiment of the invention, the compounds of the invention are administered in combination with an endothelin antagonist such as by way of example and preferably bosentan, darusentan, ambrisentan or sitaxsentan.

In a preferred embodiment of the invention, the compounds of the invention are administered in combination with a renin inhibitor such as by way of example and preferably aliskiren, SPP-600 or SPP-800.

In a preferred embodiment of the invention, the compounds of the invention are administered in combination with a mineralocorticoid receptor antagonist such as by way of example and preferably spironolactone or eplerenone.

In a preferred embodiment of the invention, the compounds of the invention are administered in combination with a Rho kinase inhibitor such as by way of example and preferably fasudil, Y-27632, SLx-2119, BF-66851, BF-66852, BF-66853, KI-23095, SB-772077, GSK-269962A or BA-1049.

In a preferred embodiment of the invention, the compounds of the invention are administered in combination with a diuretic such as by way of example and preferably furosemide.

Agents which alter lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the compounds of the invention are administered in combination with a CETP inhibitor such as by way of example and preferably torcetrapib (CP-529 414), JJT-705 or CETP vaccine (Avant).

In a preferred embodiment of the invention, the compounds of the invention are administered in combination with a thyroid receptor agonist such as by way of example and preferably D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins such as by way of example and preferably lovastatin, simvastatin, pravastatin, fluvastatin, atorvastatin, rosuvastatin, cerivastatin or pitavastatin.

In a preferred embodiment of the invention, the compounds of the invention are administered in combination with a squalene synthesis inhibitor such as by way of example and preferably BMS-188494 or TAK-475.

In a preferred embodiment of the invention, the compounds of the invention are administered in combination with an ACAT inhibitor such as by way of example and preferably avasimibe, melinamide, pactimibe, eflucimibe or SMP-797.

In a preferred embodiment of the invention, the compounds of the invention are administered in combination with an MTP inhibitor such as by way of example and preferably implitapide, BMS-201038, R-103757 or JTT-130.

In a preferred embodiment of the invention, the compounds of the invention are administered in combination with a PPAR-gamma agonist such as by way of example and preferably pioglitazone or rosiglitazone.

In a preferred embodiment of the invention, the compounds of the invention are administered in combination with a PPAR-delta agonist such as by way of example and preferably GW-501516 or BAY 68-5042.

In a preferred embodiment of the invention, the compounds of the invention are administered in combination with a cholesterol absorption inhibitor such as by way of example and preferably ezetimibe, tiqueside or pamaqueside.

In a preferred embodiment of the invention, the compounds of the invention are administered in combination with a lipase inhibitor such as by way of example and preferably orlistat.

In a preferred embodiment of the invention, the compounds of the invention are administered in combination with a polymeric bile acid adsorbent such as by way of example and preferably cholestyramine, colestipol, colesolvam, CholestaGel or colestimide.

In a preferred embodiment of the invention, the compounds of the invention are administered in combination with a bile acid reabsorption inhibitor such as by way of example and preferably ASBT (=IBAT) inhibitors such as, for example, AZD-7806, S-8921, AK-105, BARI-1741, SC-435 or SC-635.

In a preferred embodiment of the invention, the compounds of the invention are administered in combination with a lipoprotein(a) antagonist such as by way of example and preferably gemcabene calcium (CI-1027) or nicotinic acid.

The present invention further relates to medicaments comprising at least one of the compounds according to the invention, usually in combination with one or more inert, non-toxic, pharmaceutically suitable excipients, and their use for the purposes mentioned above.

The compounds of the invention may have systemic and/or local effects. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in a modified manner, and which contain the compounds of the invention in crystalline and/or amorphized and/or dissolved form, such as, for example, tablets (uncoated or coated tablets, for example having coatings which are resistant to gastric juice or are insoluble or dissolve with a delay and control the release of the compound of the invention), tablets which disintegrate rapidly in the mouth, or films/wafers, films/lyophilizates, capsules (for example hard or soft gelatin capsules), sugar-Coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other routes of administration are, for example, pharmaceutical forms for inhalation (inter alia powder inhalers, nebulizers), nasal drops, solutions or sprays, tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

Oral or parenteral administration is preferred, especially oral and intravenous administration.

The compounds of the invention can be converted into the stated administration forms. This can take place in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorings (e.g. inorganic pigments such as, for example, iron oxides) and masking flavors and/or odors.

It has generally proved to be advantageous on parenteral administration to administer amounts of about 0.001 to 1 mg/kg, preferably about 0.01 to 0.5 mg/kg of body weight to achieve effective results. On oral administration, the dosage is about 0.01 to 100 mg/kg, preferably about 0.01 to 20 mg/kg, and very particularly preferably 0.1 to 10 mg/kg of body weight.

It may nevertheless be necessary where appropriate to deviate from the stated amounts, in particular as a function of body weight, administration route, individual response to the active ingredient, type of preparation and time or interval over which administration takes place. Thus, in some cases it may be sufficient to make do with less than the aforementioned minimum amount, whereas in other cases the upper limit mentioned must be exceeded. Where relatively large amounts are administered, it may be advisable to distribute these in a plurality of single doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data of liquid/liquid solutions are based in each case on the volume.

### A. EXAMPLES

### Abbreviations:

abs. absolute
Ac acetyl
Ac₂O acetic anhydride
aq. aqueous, aqueous solution
c concentration
cat. catalytic
conc. concentrated
DCI direct chemical ionization (in MS)
DIBAH diisobutylaluminum hydride
DMF N,N-dimethylformamide
DMSO dimethyl sulfoxide
ee enantiomeric excess
EI electron impact ionization (in MS)
eq equivalent(s)
ESI electrospray ionization (in MS)
GC-MS gas chromatography-Coupled mass spectrometry
h hour(s)
HATU O-(7-azabenzotriazol-1-yl)-N,N,N′,N′-tetramethyluronium hexafluorophosphate
HPLC high pressure liquid chromatography
LC-MS liquid chromatography-Coupled mass spectrometry
m.p. melting point
Me methyl
min minute(s)
MS mass spectrometry
NBS N-bromosuccinimide
NMR nuclear magnetic resonance spectrometry
rac. racemic
RP reversed phase (in HPLC)
RT room temperature
Rₜ retention time (in HPLC)
sat. saturated
TFA trifluoroacetic acid
THF tetrahydrofuran
TLC thin-layer chromatography

HPLC, LC-MS and GC-MS Methods:

Method 1 (HPLC):

Instrument: HP 1100 with DAD detection; column: Kromasil 100 RP-18, 60 mm×2.1 mm, 3.5 μm; mobile phase A: 5 ml of HClO₄ (70% strength)/liter of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 2 (LC-MS):

Instrument: Micromass LCZ platform with HPLC Agilent series 1100; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 3 (LC-MS):

MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 4 (LC-MS):

MS instrument type: Micromass ZQ; HPLC instrument type: Waters Alliance 2795; column: Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 210 nm.

Method 5 (LC-MS):

MS instrument type: Micromass ZQ; HPLC instrument type: HP 1100 series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 6 (GC-MS):

Instrument: Micromass GCT, GC6890; column: Restek RTX-35, 15 m×200 μm×0.33 μm; constant helium flow rate: 0.88 ml/min; oven: 70° C.; inlet: 250° C.; gradient: 70° C., 30° C./min→310° C. (maintain for 3 min).

Method 7 (LC-MS):

Instrument: Micromass Quattro LCZ with HPLC Agilent series 1100; column:

Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 208-400 nm.

Method 8 (LC-MS):

MS instrument type: Micromass ZQ; HPLC instrument type: Waters Alliance 2795; column: Phenomenex Synergi 2.5μ MAX-RP 100A Mercury, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 9 (LC-MS):

Instrument: Micromass Quattro LCZ with HPLC Agilent series 1100; column: Phenomenex Synergi 2.5μ MAX-RP 100A Mercury, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

Starting materials and Intermediates:

### Example 1A

### 2-Amino-5-phenyl-3-furonitrile

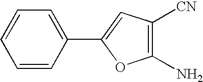

At RT, 68.6 ml (663 mmol) of diethylamine were added dropwise (cooling required to maintain the temperature) to a mixture of 60.0 g (301 mmol) of bromoacetophenone and 25.89 g (391.86 mmol) of malononitrile in 130 ml of DMF. Cooling was removed toward the end of the addition, and the mixture was stirred at RT for 1 h and then poured into 385 ml of water. The mixture was diluted with a further 125 ml of water and stirred at RT for 20 min. The precipitated solid was filtered off with suction, washed twice with in each case 125 ml of water, filtered off with suction to dryness and washed with petroleum ether. The residue was dried under high vacuum. This gave 33.3 g (50.1% of theory) of the target compound as crystals.

HPLC (method 1): Rₜ=4.27 min

MS (DCI): m/z=202 (M+NH₄)⁺, 185 (M+H)⁺

¹H-NMR (400 MHz, CDCl₃): δ=7.51-7.45 (m, 2H), 7.39-7.32 (m, 3H), 6.54 (s, 1H), 4.89 (br. s, 1H).

### Example 2A

### 6-Phenylfuro[2,3-d]pyrimidine-4-amine

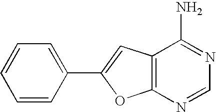

110 g (597 mmol) of 2-amino-5-phenyl-3-furonitrile were suspended in 355 ml (9 mol) of formamide and heated for 1.5 h (bath temperature about 210° C.). The mixture was then cooled to RT and stirred into water. The precipitated solid was filtered off with suction and washed with water. The product, which was still moist, was triturated with dichloromethane, once more filtered off with suction and dried under reduced pressure.

This gave 106 g (80% of theory) of the target compound.

LC-MS (method 2): Rₜ=3.1 min; m/z 32 212 (M+H)⁺

HPLC (method 1): Rₜ=3.63 min.

¹H-NMR (400 MHz, DMSO-d₆): δ=8.20 (s, 1H), 7.8 (d, 2H), 7.55-7.32 (m, 6H).

### Example 3A

### 5-Bromo-6-phenylfuro[2,3-d]pyrimidine-4-amine

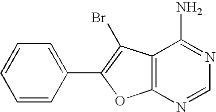

In 770 ml of carbon tetrachloride, 80 g (378.7 mmol) of 6-phenylfuro[2,3-d]pyrimidine-4-amine were heated to 60° C. 84.3 g (473.4 mmol) of N-bromosuccinimide were added, and the mixture was stirred under reflux overnight. After cooling, the mixture was filtered off, and the filter cake was triturated successively with dichloromethane and acetonitrile and filtered off again. The filter cake was then dried under reduced pressure. This gave 86 g of the target product (78.2% of theory).

MS (DCI): m/z=290/292 (M+H)⁺

¹H-NMR (400 MHz, DMSO-d₆): δ=8.28 (s, 1H), 8.03 (d, 2H), 7.60-7.50 (m, 5H).

### Example 4A

### 5-Bromo-4-Chloro-6-phenylfuro[2,3-d]pyrimidine

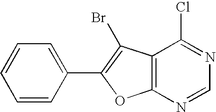

54 g (186 mmol) of 5-bromo-6-phenylfuro[2,3-d]pyrimidine-4-amine were initially charged in 135 ml of chloroform, 70 ml of 4 N hydrogen chloride in dioxane (280 mmol) were added and the mixture was heated at reflux. With evolution of gas, 50 ml (372 mmol) of isoamyl nitrite were added dropwise. After the addition had ended, the mixture was stirred at reflux for 3 h, and the cooled reaction mixture was then added to water and extracted with dichloromethane. The organic phase was washed with sat. sodium bicarbonate solution, dried over sodium sulfate and concentrated under reduced pressure. The crude product was purified by chromatography on silica gel (mobile phase: dichloromethane). For further purification, the product was triturated with methanol, filtered off with suction and dried under high vacuum. This gave 32 g of the target product (55.5% of theory).

LC-MS (method 3): Rₜ=2.54 min; m/z=309/310 (M+H)⁺

HPLC (method 1): Rₜ=5.08 min.

¹H-NMR (400 MHz, CDCl₃): δ=8.79 (s, 1H), 8.23-8.20 (m, 2H), 7.58-7.51 (m, 3H).

### Example 5A

### tert-Butyl (2E,6R)-6-hydroxyhept-2-enoate

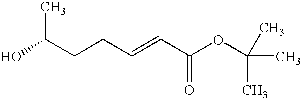

Solution A: 10.71 g (267.7 mmol) of 60% pure sodium hydride were suspended in 150 ml of abs. THF, and 43.3 ml (276.7 mmol) of tert-butyl P,P-dimethylphosphonoacetate were added dropwise with cooling. The mixture was stirred at RT, and after about 30 min a solution had formed.

187.4 ml (187.4 mmol) of a 1 M solution of DIBAH in THF were added dropwise to a solution, cooled to −78° C., of 17.87 g (178.5 mmol) of (R)-γ-valerolactone [(5R)-5-methyl-dihydrofuran-2(3H)-one] in 200 ml of abs. THF. The solution was stirred at −78° C. for 1 h, and solution A, prepared above, was then added. After the addition had ended, the mixture was slowly warmed to RT and stirred at RT overnight. The reaction mixture was then added to 300 ml of ethyl acetate and extracted with 50 ml of concentrated potassium sodium tartrate solution. After phase separation, the aqueous phase was reextracted with ethyl acetate. The organic phases were combined, washed with sat. sodium chloride solution, dried over magnesium sulfate and concentrated under reduced pressure. The residue was purified by chromatography on silica gel (mobile phase: cyclohexane/ethyl acetate 5:1). This gave 32.2 g (90.1% of theory) of the target product, which contained small amounts of the cis-isomer.

MS (DCI): m/z=218 (M+NH₄)⁺

¹H-NMR (400 MHz, DMSO-d₆): δ=6.70 (dt, 1H), 5.73 (d, 1H), 4.44 (d, 1H), 3.58 (m, 1H), 2.28-2.13 (m, 2H), 1.47-1.40 (m, 2H), 1.45 (s, 9H), 1.04 (d, 3H).

### Example 6A

### tert-Butyl (−)-6-hydroxyheptanoate

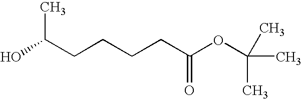

32.2 g (160.8 mmol) of tert-butyl (2E,6R)-6-hydroxyhept-2-enoate were dissolved in 200 ml of ethanol, and 1.7 g of 10% palladium on carbon were added. At RT, the mixture was stirred under an atmosphere of hydrogen (atmospheric pressure) for 2 h and then filtered off through Celite. The filtrate was concentrated under reduced pressure. The residue gave, after chromatography on silica gel (mobile phase: cyclohexane/ethyl acetate 10:1→6:1), 15.66 g of the target product (48.1% of theory).

MS (DCI): m/z=220 (M+NH₄)⁺

¹H-NMR (400 MHz, CDCl₃): δ=3.85-3.75 (m, 1H), 2.22 (t, 2H), 1.68-1.54 (m, 2H), 1.53-1.30 (m, 4H), 1.45 (s, 9H), 1.18 (d, 3H).

[α]_{D}²⁰=−21°, c=0.118, chloroform.

### Example 7A

### tert-Butyl (6R)-6-[(5-bromo-6-phenylfuro[2,3-d]pyrimidin-4-yl)oxy]heptanoate

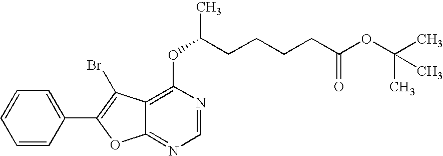

10.0 g (32.30 mmol) of 5-bromo-4-Chloro-6-phenylfuro[2,3-d]pyrimidine and 10.8 g (53.30 mmol) of tert-butyl (−)-6-hydroxyheptanoate were initially charged in 20 ml of DMF, and 2.1 g (53.30 mmol) of 60% pure sodium hydride were added at 0° C. The reaction mixture was then warmed to RT, and stirring at this temperature was continued for 45 min. Water was then added, and the reaction mixture was extracted with dichloromethane. The organic phase was washed with saturated sodium chloride solution, dried over sodium sulfate and concentrated under reduced pressure. The residue was chromatographed on silica gel using a gradient of cyclohexane and ethyl acetate (20:1→10:1). This gave 6.8 g of the target product (44% of theory).

LC-MS (method 4): Rₜ=4.87 min; m/z=475 (M+H)⁺

¹H-NMR (400 MHz, DMSO-d₆): δ=8.60 (s, 1H), 8.06 (d, 2H), 7.64-7.50 (m, 3H), 5.48 (m, 1H), 2.18 (t, 2H), 1.76 (m, 2H), 1.61-1.28 (m, 7H), 1.33 (s, 9H).

[α]_{D}²⁰=56°, c=0.450, chloroform.

### Example 8A

### tert-Butyl (6R)-6-[(6-phenyl-5-vinylfuro[2,3-d]pyrimidin-4-yl)oxy]heptanoate

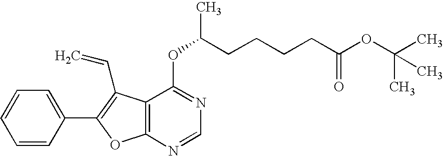

Under an atmosphere of argon, 3.0 g (6.31 mmol) of tert-butyl (6R)-6-[(5-bromo-6-phenylfuro[2,3-d]pyrimidin-4-yl)oxy]heptanoate were dissolved in 20 ml of THF, and 6.3 ml of 2 N aqueous sodium carbonate solution were added. After addition of 1.458 g (9.47 mmol) of 2-vinylboronic acid pinacol ester and 0.443 g (0.63 mmol) of bis(triphenylphosphine)palladium(II) chloride, the mixture was stirred under reflux overnight. After cooling, the reaction mixture was filtered through Celite, the filtrate was concentrated and the residue was dried under high vacuum. The crude product was purified by chromatography on silica gel (mobile phase: cyclohexane/ethyl acetate 10:1→8:1). This gave 2.08 g of the target product (70.8% of theory).

LC-MS (method 5): Rₜ=3.58 min; m/z=423 (M+H)⁺

¹H-NMR (400 MHz, DMSO-d₆): δ=8.61 (s, 1H), 7.37 (d, 2H), 7.60-7.50 (m, 3H), 6.39 (dd, 1H), 6.28 (dd, 1H), 5.59 (dd, 1H), 5.52 (m, 1H), 2.19 (t, 2H), 1.85-1.69 (m, 2H), 1.58-1.48 (m, 2H), 1.45-1.37 (m, 2H), 1.39 (d, 3H), 1.32 (s, 9H).

[α]_{D}²⁰=47.4°, c=0.330, chloroform.

### Example 9A

### tert-Butyl (6R)-6-[(5-formyl-6-phenylfuro[2,3-d]pyrimidin-4-yl)oxy]heptanoate

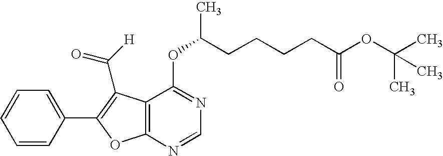

1.55 g (3.55 mmol) of tert-butyl (6R)-6-[(6-phenyl-5-vinylfuro[2,3-d]pyrimidin-4-yl)oxy]heptanoate were dissolved in 17.3 ml of methanol and 17.3 ml of dichloromethane and cooled to −78° C. Ozone gas was generated in an ozonizer and, in a stream of ozygen, passed for about 10 min through the reaction mixture, the color of which changed to green-blue. The ozonizer was switched off, and excess ozone was flushed from the reaction mixture by the gas stream. 8 ml of dimethyl sulfoxide were then added to the reaction mixture, which was still cold and light-green, and the mixture was slowly warmed to RT. The mixture was then concentrated and the residue was dried under high vaccum. The crude product was purified by chromatography on silica gel (mobile phase: cyclohexane/ethyl acetate 10:1). This gave 0.81 g of the target product (53.1% of theory).

LC-MS (method 5): Rₜ=3.31 min; m/z=425 (M+H)⁺

¹H-NMR (400 MHz, DMSO-d₆): δ=10.32 (s, 1H), 8.68 (s, 1H), 8.65 (d, 2H), 7.68-7.60 (m, 3H), 5.52 (m, 1H), 2.19 (t, 2H), 1.89-1.70 (m, 2H), 1.58-1.50 (m, 2H), 1.48-1.38 (m, 2H), 1.42 (d, 3H), 1.35 (s, 9H).

[α]_{D}²⁰=52°, c=0.460, chloroform.

### Example 10A

### N-Ethyl-2-methylacrylamide

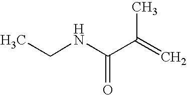

0.50 g (5.8 mmol) of methacrylic acid was dissolved in 4 ml of DMF and cooled to 0° C., and 4.42 g (11.62 mmol) of HATU were added. The mixture was stirred at 0° C. for 10 min, after which 2.0 ml (11.62 mmol) of N,N-diisopropylethylamine and 8.7 ml (17.4 mmol) of a 2 M solution of ethylamine in methanol were added. The reaction mixture was warmed to RT and stirred overnight. The mixture was then diluted with ethyl acetate and washed with water and sat. sodium chloride solution. The organic phase was dried over sodium sulfate and concentrated, and the residue was dried under high vacuum. The crude product was purified by chromatography on silica gel (mobile phase: dichloromethane/methanol 100:1). To remove any residual DMF and N,N-diiso-propylethylamine, the product obtained was taken up in ethyl acetate and washed three times with sat. ammonium chloride solution. The organic phase was then dried over sodium sulfate and concentrated under reduced pressure, and the residue was dried thoroughly under high vaccum. This gave 0.91 g of the target product (purity about 65%, 90.7% of theory).

GC-MS (method 6): Rₜ=2.59 min; m/z=113 (M⁺).

### Example 11A

### N-Propyl-2-methylacrylamide

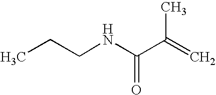

The title compound was prepared in a manner analogous to the procedure of Example 10A.

GC-MS (method 6): Rₜ=3.01 min; m/z=127 (M⁺).

### WORKING EXAMPLES

### Example 1

### tert-Butyl (6R)-6-({5-[(1E)-pent-1-en-1-yl]-6-phenylfuro[2,3-d]pyrimidin-4-yl}oxy)heptanoate

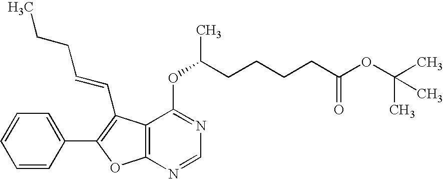

Under an atmosphere of argon, 100 mg (0.21 mmol) of tert-butyl (6R)-6-[(5-bromo-6-phenylfuro[2,3-d]pyrimidin-4-yl)oxy]heptanoate were dissolved in 2.0 ml of THF, and 1.05 ml of 2 N aqueous sodium carbonate solution, 53.9 mg (0.47 mmol) of 1-pentenylboronic acid and 14.8 mg (0.021 mmol) of bis(triphenylphosphine)palladium(II) chloride were added in succession. The mixture was stirred under reflux overnight and then, after cooling, filtered through Celite. The filtrate was concentrated and the residue was purified chromatographically on silica gel (mobile phase: cyclohexane/ethyl acetate 10:1). This gave 73.9 mg of the target product (75.0% of theory).

LC-MS (method 4): Rₜ=5.22 min; m/z=465 (M+H)⁺

¹H-NMR (400 MHz, DMSO-d₆): δ=8.57 (s, 1H), 7.78 (d, 2H), 7.60-7.48 (m, 3H), 6.69 (dt, 1H), 6.53 (d, 1H), 5.53 (m, 1H), 2.25 (q, 2H), 2.19 (t, 2H), 1.79-1.65 (m, 3H), 1.55-1.45 (m, 3H), 1.39 (d, 3H), 1.35 (s, 9H), 0.96 (t, 3H).

[α]_{D}²⁰=−49°, c=0.225, chloroform.

### Example 2

### tert-Butyl (6R)-6-({5-[(1E)-3-oxopent-1-en-1-yl]-6-phenylfuro[2,3-d]pyrimidin-4-yl}oxy)heptanoate

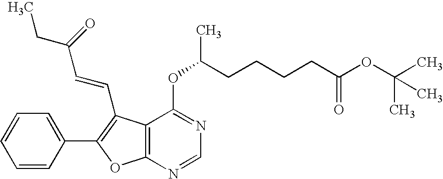

53.9 mg (0.259 mmol) of diethyl (2-oxobutane)phosphonate were dissolved in 4.0 ml of THF and cooled to 0° C., and 10.3 mg (0.259 mmol, 60% pure) of sodium hydride were added. The mixture was stirred for 5 min, and 100 mg (0.236 mmol) of tert-butyl (6R)-6-[(5-formyl-6-phenylfuro[2,3-d]pyrimidin-4-yl)oxy]heptanoate were then added. After warming to RT, stirring of the reaction mixture was continued overnight. Water was then added, and the mixture was extracted with ethyl acetate. The organic phase was washed with sat. sodium chloride solution, dried over sodium sulfate and concentrated under reduced pressure. The crude product was purified by preparative RP-HPLC (mobile phase: acetonitrile/water gradient). This gave 81.2 mg of the target product (72% of theory).

LC-MS (method 5): Rₜ=3.44 min; m/z=479 (M+H)⁺

¹H-NMR (400 MHz, DMSO-d₆): δ=8.68 (s, 1H), 7.78 (d, 2H), 7.70-7.58 (m, 3H), 7.43 (d, 2H), 5.07 (m, 1H), 2.75-2.67 (m, 2H), 2.19 (t, 2H), 1.90-1.75 (m, 2H), 1.57-1.50 (m, 2H), 1.44 (d, 3H), 1.35 (s, 9H), 1.05 (t, 3H).

[α]_{D}²⁰=−61°, c=0.11, chloroform.

### Example 3

### tert-Butyl (6R)-6-({5-[(1E)-3-(ethoxy)-3-oxoprop-1-en-1-yl]-6-phenylfuro[2,3-d]pyrimidin-4-yl}oxy)heptanoate

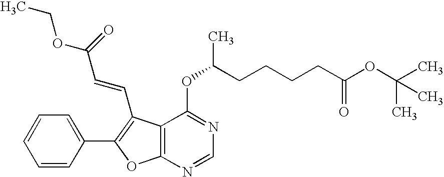

29.1 mg (0.13 mmol) of triethyl phosphonoacetate were dissolved in 2.0 ml of THF and cooled to 0° C., and 5.2 mg (0.13 mmol, 60% pure) of sodium hydride were added. The mixture was stirred for 5 min, and 50 mg (0.118 mmol) of tert-butyl (6R)-6-[(5-formyl-6-phenylfuro[2,3-d]pyrimidin-4-yl)oxy]heptanoate were then added. After warming to RT, stirring of the reaction mixture was continued overnight. Water was then added, and the mixture was extracted with ethyl acetate. The organic phase was washed with sat. sodium chloride solution, dried over sodium sulfate and concentrated under reduced pressure. The crude product was purified by preparative RP-HPLC (mobile phase: acetonitrile/water gradient). This gave 35.0 mg of the target product (60.1% of theory).

LC-MS (method 7): Rₜ=4.99 min; m/z=495 (M+H)⁺

¹H-NMR (400 MHz, DMSO-d₆): δ=8.67 (s, 1H), 7.78-7.61 (m, 6H), 7.16 (d, 1H), 5.50 (m, 1H), 4.23-4.17 (m, 2H), 2.18 (t, 2H), 1.86-1.75 (m, 2H), 1.60-1.51 (m, 2H), 1.41 (d, 3H), 1.34 (s, 9H), 1.26 (t, 3H).

[α]_{D}²⁰=56°, c=0.225, chloroform.

### Example 4

### tert-Butyl (6R)-6-({5-[(1E)-3-(allyloxy)-3-oxoprop-1-en-1-yl]-6-phenylfuro[2,3-d]pyrimidin-4-yl}oxy)heptanoate

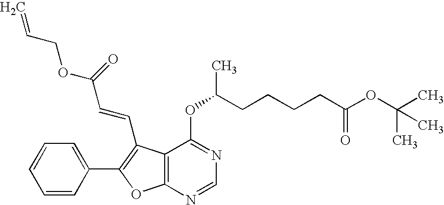

122.4 mg (0.518 mmol) of allyl diethylphosphonoacetate were dissolved in 8.0 ml of THF and cooled to 0° C., and 20.3 mg (0.518 mmol, 60% pure) of sodium hydride were added. The mixture was stirred for 5 min, and 200 mg (0.471 mmol) of tert-butyl (6R)-6-[(5-formyl-6-phenylfuro[2,3-d]pyrimidin-4-yl)oxy]heptanoate were then added. After warming to RT, stirring of the reaction mixture was continued overnight. Water was then added, and the mixture was extracted with ethyl acetate. The organic phase was washed with sat. sodium chloride solution, dried over sodium sulfate and concentrated under reduced pressure. The crude product was purified by preparative RP-HPLC (mobile phase: acetonitrile/water gradient). This gave 225.1 mg of the target product (94.3% of theory).

LC-MS (method 5): Rₜ=3.53 min; m/z=507 (M+H)⁺

¹H-NMR (400 MHz, DMSO-d₆): δ=8.67 (s, 1H), 7.78-7.61 (m, 6H), 7.20 (d, 1H), 6.40-5.95 (m, 1H), 5.52 (m, 1H), 5.35 (dd, 1H), 5.27 (dd, 1H), 4.69 (d, 2H), 2.18 (t, 2H), 1.85-1.75 (m, 2H), 1.59-1.50 (m, 2H), 1.48-1.36 (m+t, together 5H), 1.33 (s, 9H).

[α]_{D}²⁰=56°, c=0.100, chloroform.

### Example 5

### (2E)-3-(4-{[(1R)-6-tert-Butoxy-1-methyl-6-oxohexyl]oxy}-6-phenylfuro[2,3-d]pyrimidin-5-yl)acrylic acid

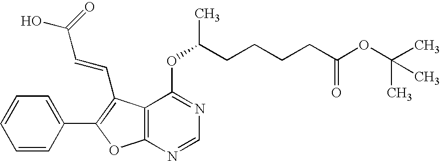

Under argon, 220.0 mg (0.46 mmol) of tert-butyl (6R)-6-({5-[(1E)-3-(allyloxy)-3-oxoprop-1-en-1-yl]-6-phenylfuro[2,3-d]pyrimidin-4-yl}oxy)heptanoate and 57 l (0.643 mmol) of morpholine were dissolved in 7.0 ml of THF, and 5.0 mg (0.004 mmol) of tetrakis(triphenylphosphine)palladium(0) were added. The reaction mixture was stirred at RT for 3 h and then filtered through Celite. The filter residue was washed with dichloromethane, and the combined filtrates were washed with water and sat. sodium chloride solution. The organic phase was dried over sodium sulfate and concentrated under reduced pressure. The residue was purified by preparative RP-HPLC (mobile phase: acetonitrile/water gradient). This gave 182 mg of the target product (85.0% of theory).

LC-MS (method 8): Rₜ=2.51 min; m/z=467 (M+H)⁺

¹H-NMR (400 MHz, DMSO-d₆): δ=12.55 (br. s, 1H), 8.55 (s, 1H), 7.75 (d, 2H), 7.69-7.57 (m, 4H), 7.05 (d, 1H), 5.54 (m, 1H), 2.18 (t, 2H), 1.88-1.72 (m, 2H), 1.55-1.50 (m, 2H), 1.45-1.37 (m+t, together 5H), 1.33 (s, 9H).

[α]_{D}²⁰=49°, c=0.175, chloroform.

### Example 6

### tert-Butyl (6R)-6-({5-[(1E)-3-ethoxy-2-methyl-3-oxoprop-1-en-1-yl]-6-phenylfuro[2,3-d]-pyrimidin-4-yl}oxy)heptanoate

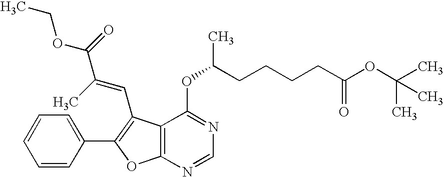

Under an atmosphere of argon, 100 mg (0.21 mmol) of tert-butyl (6R)-6-[(5-bromo-6-phenylfuro[2,3-d]pyrimidin-4-yl)oxy]heptanoate, 0.13 ml (1.052 mmol) of ethyl methacrylate, 13.6 mg (0.042 mmol) of tetra-n-butylammonium bromide, 0.55 ml (3.16 mmol) of N,N-diisopropylethylamine and 6.6 mg (0.008 mmol) of dichlorobis(tri-o-tolylphosphine)palladium(II) were mixed in 3.0 ml of DMF and heated at 110° C. overnight. After cooling to RT, the reaction mixture was diluted with ethyl acetate, washed with water and sat. sodium chloride solution, dried over sodium sulfate and concentrated. The residue was dried under high vacuum and the crude product was purified by preparative RP-HPLC (mobile phase: water/acetonitrile gradient). This gave 51.9 mg of the target product (48.5% of theory).

LC-MS (method 8): Rₜ=3.06 min; m/z=509 (M+H)⁺

1H-NMR (400 MHz, DMSO-d₆): δ=8.61 (s, 1H), 7.74 (d, 2H), 7.60-7.47 (m, 3H), 5.39 (m, 1H), 4.25 (q, 2H), 2.16 (t, 2H), 1.71-1.65 (m, 2H), 1.65 (s, 3H), 1.54-1.43 (m, 2H), 1.40-1.29 (m, 17H).

[α]_{D}²⁰=−51.2°, c=0.365, chloroform.

### Example 7

### tert-Butyl (6R)-6-({5-[(1E)-3-oxohex-1-en-1-yl]-6-phenylfuro[2,3-d]pyrimidin-4-yl}oxy)-heptanoate

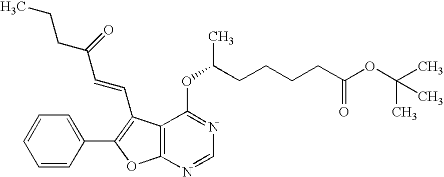

Under an atmosphere of argon, 100 mg (0.21 mmol) of tert-butyl (6R)-6-[(5-bromo-6-phenylfuro[2,3-d]pyrimidin-4-yl)oxy]heptanoate, 103.2 mg (1.052 mmol) of 1-hexen-3-one, 13.6 mg (0.042 mmol) of tetra-n-butylammonium bromide, 0.44 ml (3.16 mmol) of triethylamine and 6.6 mg (0.008 mmol) of dichlorobis(tri-o-tolylphosphine)palladium(II) were mixed in 3.0 ml of DMF and heated to 110° C. After 4 h, a further 0.04 eq. of dichlorobis(tri-o-tolylphosphine)palladium(II) and 0.55 ml (3.16 mmol) of N,N-diisopropylethylamine were added to the reaction mixture. The mixture was then stirred at 110° C. overnight. After cooling to RT, the reaction mixture was diluted with ethyl acetate, washed with water and sat. sodium chloride solution, dried over sodium sulfate and concentrated. The residue was dried under high vacuum and the crude product was purified by preparative RP-HPLC (mobile phase: water/acetonitrile gradient). This gave 51.9 mg of the target product (48.5% of theory).

LC-MS (method 5): Rₜ=3.51 min; m/z=493 (M+H)⁺

¹H-NMR (400 MHz, DMSO-d₆): δ=8.67 (s, 1H), 7.78 (d, 2H), 7.69-7.59 (m, 4H), 7.41 (d, 1H), 5.56 (m, 1H), 2.65 (dt, 2H), 2.19 (t, 2H), 1.90-1.75 (m, 2H), 1.64-1.51 (m, 4H), 1.49-1.36 (m, 2H), 1.45 (d, 3H), 1.32 (s, 9H), 0.91 (t, 3H).

[α]_{D}²⁰=−60°, c=0.115, chloroform.

### Example 8

### tert-Butyl (6R)-6-({5-[(1E)-3-(ethylamino)-3-oxoprop-1-en-1-yl]-6-phenylfuro[2,3-d]pyrimidin-4-yl}oxy)heptanoate

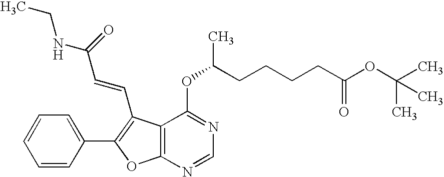

50 mg (0.107 mmol) of (2E)-3-(4-{[(1R)-6-tert-butoxy-1-methyl-6-oxohexyl]oxy}-6-phenylfuro[2,3-d]pyrimidin-5-yl)acrylic acid were dissolved in 1.0 ml of DMF and cooled to 0° C., and 81.5 mg (0.214 mmol) of HATU were added. The mixture was stirred at 0° C. for 10 min, and 37 l (0.214 mmol) of N,N-diisopropylethylamine and 161 l (0.214 mmol) of a 2 M solution of ethylamine in methanol were then added. The reaction mixture was stirred at RT overnight and then diluted with ethyl acetate and washed with water and sat. sodium chloride solution. The organic phase was dried over sodium sulfate and concentrated, and the residue was dried under high vacuum. The crude product was purified by chromatography on silica gel (mobile phase: dichloro-methane/methanol 200:1). This gave 38.4 mg of the target product (72.6% of theory).

LC-MS (method 9): Rₜ=2.70 min; m/z=494 (M+H)⁺

¹H-NMR (400 MHz, DMSO-d₆): δ=8.61 (s, 1H), 8.12 (t, 1H), 7.75 (d, 2H), 7.65-7.52 (m, 4H), 6.93 (d, 1H), 5.44 (m, 1H), 3.29-3.18 (m, 2H), 2.18 (t, 2H), 1.98-1.89 (m, 1H), 1.78-1.69 (m, 1H), 1.55-1.48 (m, 2H), 1.47 (d, 3H), 1.42-1.35 (m, 2H), 1.35 (s, 9H), 1.10 (t, 3H).

[α]_{D}²⁰=49°, c=0.15, chloroform.

### Example 9

### tert-Butyl (6R)-6-({5-[(1E)-3-(allylamino)-3-oxoprop-1-en-1-yl]-6-phenylfuro[2,3-d]pyrimidin-4-yl}oxy)heptanoate

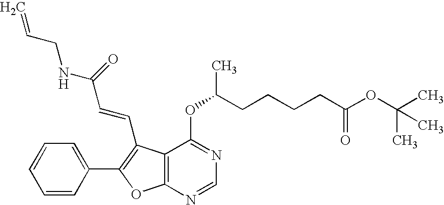

The title compound was obtained in a manner analogous to the procedure of Example 8.

LC-MS (method 5): Rₜ=3.18 min; m/z=506 (M+H)⁺

¹H-NMR (400 MHz, DMSO-d₆): δ=8.63 (s, 1H), 8.34 (t, 1H), 7.73 (d, 2H), 7.66-7.54 (m, 4H), 6.99 (d, 1H), 5.93-5.84 (m, 1H), 5.46 (m, 1H), 5.20-5.09 (m, 2H), 3.91-3.79 (m, 2H), 2.18 (t, 2H), 1.97-1.89 (m, 1H), 1.88-1.70 (m, 1H), 1.53-1.48 (m, 2H), 1.48 (d, 3H), 1.41-1.35 (m, 2H), 1.35 (s, 9H).

[α]_{D}²⁰=−58°, c=0.105, chloroform.

### Example 10

### tert-Butyl (6R)-6-({5-[(1E)-3-(ethylamino)-2-methyl-3-oxoprop-1-en-1-yl]-6-phenylfuro[2,3-d]pyrimidin-4-yl}oxy)heptanoate

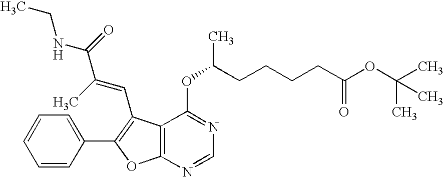

Under an atmosphere of argon, 2500 mg (0.53 mmol) of tert-butyl (6R)-6-[(5-bromo-6-phenylfuro[2,3-d]pyrimidin-4-yl)oxy]heptanoate, 457 mg (65% pure, 2.63 mmol) of N-ethyl-2-methylacrylamide, 33.9 mg (0.105 mmol) of tetra-n-butylammonium bromide, 1.4 ml (7.9 mmol) of N,N-diisopropylethylamine and 15.6 mg (0.021 mmol) of dichlorobis(tri-o-tolylphosphine)palladium(II) were mixed in 5.0 ml of DMF and heated at 110° C. overnight. After cooling to RT, the reaction mixture was diluted with ethyl acetate, washed with water and sat. sodium chloride solution, dried over sodium sulfate and concentrated. The residue was dried under hight vacuum and the crude product was purified by preparative HPLC (column: Daicel Chiralpak IA 5 m, 250 mm×20 mm; flow rate: 15 ml/min; temperature: 30° C.; mobile phase: methyl tert-butyl ether/acetonitrile 80:20). This gave 30 mg of the target product (11.2% of theory).

¹H-NMR (500 MHz, DMSO-d₆): δ=8.58 (s, 1H), 8.02 (t, 1H), 7.78 (d, 2H), 7.57-7.45 (m, 3H), 7.37 (s, 1H), 5.36 (m, 1H), 3.30-3.23 (m, 2H), 2.17 (t, 2H), 1.70-1.62 (m, 2H), 1.68 (s, 3H), 1.52-1.47 (m, 2H), 1.39-1.31 (m, 14H), 1.11 (t, 3H).

[α]_{D}²⁰=58°, c=0.050, chloroform.

### Example 11

### tert-Butyl (6R)-6-({5-[(1E)-2-methyl-3-oxo-3-(propylamino)prop-1-en-1-yl]-6-phenylfuro[2,3-d]pyrimidin-4-yl}oxy)heptanoate

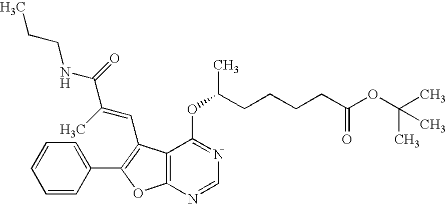

The title compound was obtained in a manner analogous to the procedure of Example 8.

Yield: 26 mg (9.5% of theory)

¹H-NMR (500 MHz, DMSO-d₆): δ=8.58 (s, 1H), 8.03 (t, 1H), 7.79 (d, 2H), 7.57-7.45 (m, 3H), 7.38 (s, 1H), 5.38 (m, 1H), 3.28-3.18 (m, 2H), 2.16 (t, 2H), 1.71-1.62 (m, 2H), 1.68 (s, 3H), 1.58-1.45 (m, 4H), 1.39-1.30 (m, 14H), 1.10 (t, 3H).

[α]_{D}²⁰=50°, c=0.050, chloroform.

General Procedure A: Cleavage of tert-butyl esters to give the corresponding carboxylic acids

At 0° C. to RT, trifluoroacetic acid (TFA) is added dropwise to a solution of the tert-butyl ester in dichloromethane (concentration 0.1 to 1.0 mol/l; additionally optionally a drop of water) until a dichloromethane/TFA ratio of about 2:1 to 1:2 is reached. The mixture is stirred at RT for 1-18 h and then concentrated under reduced pressure. Alternatively, the reaction mixture is diluted with dichloromethane, washed with water and sat. sodium chloride solution, dried and concentrated under reduced pressure. If required, the crude product can be purified, for example by preparative RP-HPLC (mobile phase: acetonitrile/water gradient).

The following Working Examples were obtained according to General Procedure A:

| Example | Structure | Analytical data |
|---|---|---|
| 12 | 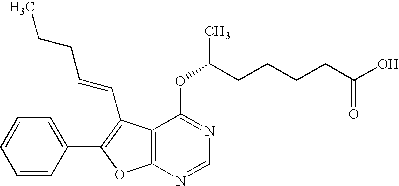 | LC-MS (method 4): Rₜ = 4.32 min; m/z = 409 (M + H)⁺ ¹H-NMR (400 MHz, DMSO-d₆): δ = 11.97 (br. s, 1H), 8.59 (s, 1H), 7.77 (d, 2H), 7.60-7.48 (m, 3H), 6.68 (dt, 1H), 6.52 (d, 1H), 5.51 (m, 1H), 2.29-2.19 (m, 4H), 1.82-1.70 (m, 2H), 1.60-1.49 (m, 6H), 1.38 (d, 3H), 0.95 (t, 3H). [α]_{D}²⁰ = −69° , c = 0.280, chloro- form. |
| 13 | 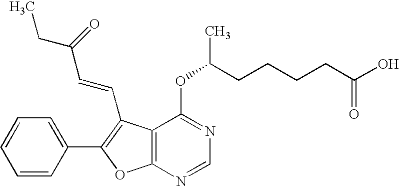 | LC-MS (method 2): Rₜ = 3.91 min; m/z = 423 (M + H)⁺ ¹H-NMR (400 MHz, DMSO-d₆): δ = 11.98 (s, 1H), 8.68 (s, 1H), 7.77 (d, 2H), 7.68-7.60 (m, 4H), 7.43 (d, 1H), 5.55 (m, 1H), 2.71 (q, 2H), 2.22 (t, 2H), 1.92-1.73 (m, 2H), 1.59-1.39 (m, 4H), 1.41 (d, 3H), 1.04 (t, 3H). [α]_{D}²⁰ = −61°, c = 0.120, chloro- form. |
| 14 | 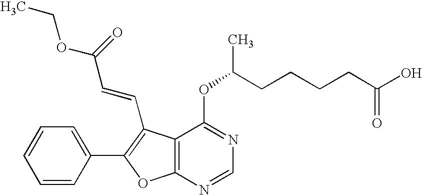 | LC-MS (method 7): R^{t} = 4.04 min; m/z = 439 (M + H)⁺ ¹H-NMR (400 MHz, DMSO-d₆): δ = 11.98 (br. s, 1H), 8.67 (s, 1H), 7.78-7.60 (m, 6H), 7.18 (d, 1H), 5.50 (m, 1H), 4.19 (q, 2H), 2.21 (t, 1H), 1.88-1.73 (m, 2H), 1.60-1.40 (m, 4H), 1.42 (d, 3H), 1.28 (t, 3H). [α]_{D}²⁰ = −59°, c = 0.235, chloro- form. |
| 15 | 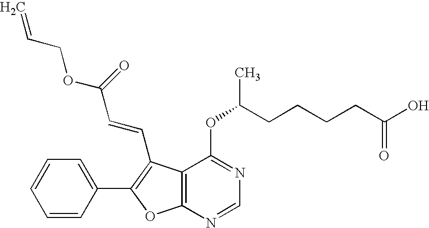 | LC-MS (method 5): Rₜ = 2.97 min; m/z = 451 (M + H)⁺ ¹H-NMR (400 MHz, DMSO-d₆): δ = 11.98 (br. s, 1H), 8.69 (s, 1H), 7.78-7.63 (m, 6H), 7.71 (d, 1H), 6.04-5.95 (m, 1H), 5.51 (m, 1H), 5.36 (dd, 1H), 5.28 (dd, 1H), 4.70 (d, 2H), 2.18 (t, 2H), 1.89-1.72 (m, 2H), 1.60-1.40 (m, 4H), 1.42 (d, 3H). [α]_{D}²⁰ = −43°, c = 0.190, chloro- form. |
| 16 | 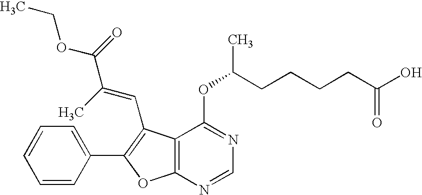 | LC-MS (method 8): Rₜ = 2.33 min; m/z = 453 (M + H)⁺ ¹H-NMR (400 MHz, DMSO-d₆): δ = 11.97 (s, 1H), 8.60 (s, 1H), 7.78-7.72 (m, 3H), 7.60-7.48 (m, 3H), 5.39 (m, 1H), 4.25 (q, 2H), 2.20 (t, 2H), 1.72-1.65 (m, 2H), 1.65 (s, 3H), 1.55-1.45 (m, 2H), 1.41-1.29 (m, 8H). [α]_{D}²⁰ = −33°, c = 0.075, chloro- form. |
| 17 | 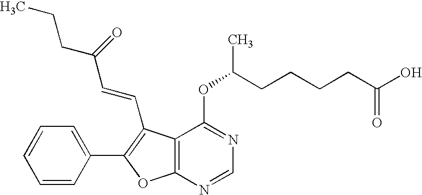 | LC-MS (method 5): Rₜ = 2.90 min; m/z = 437 (M + H)⁺ ¹H-NMR (400 MHz, DMSO-d₆): δ = 11.98 (s, 1H), 8.67 (s, 1H), 7.77 (d, 2H), 7.69-7.59 (m, 4H), 7.41 (d, 1H), 5.58 (m, 1H), 2.66 (t, 2H), 2.22 (t, 2H), 1.92-1.73 (m, 2H), 1.65-1.40 (m, 6H), 1.45 (d, 3H), 0.92 (t, 3H). [α]_{D}²⁰ = −39°, c = 0.090, chloro- form. |
| 18 | 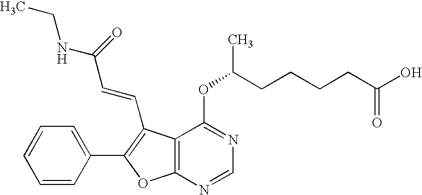 | LC-MS (method 5): Rₜ = 2.42 min; m/z = 438 (M + H)⁺ ¹H-NMR (400 MHz, DMSO-d₆): δ = 11.97 (s, 1H), 8.65 (s, 1H), 8.12 (t, 1H), 7.73 (d, 2H), 7.66- 7.52 (m, 4H), 6.92 (d, 1H), 5.43 (m, 1H), 3.28-3.18 (m, 2H), 2.22 (t, 2H), 1.98-1.89 (m, 1H), 1.80- 1.68 (m, 1H), 1.58-1.49 (m, 2H), 1.45 (d, 3H), 1.44-1.20 (m, 2H), 1.11 (t, 3H). [α]_{D}²⁰ = −37°, c = 0.08, chloro- form. |
| 19 | 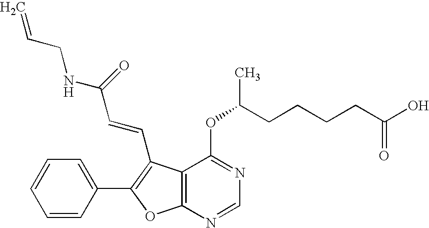 | LC-MS (method 8): Rₜ = 1.91 min; m/z = 450 (M + H)⁺ ¹H-NMR (400 MHz, DMSO-d₆): δ = 11.97 (br. s, 1H), 8.63 (s, 1H), 8.36 (m, 1H), 7.72 (d, 2H), 7.65-7.55 (m, 4H), 7.01 (d, 1H), 5.92-5.83 (m, 1H), 5.45 (m, 1H), 5.18 (dd, 1H), 5.11 (dd, 1H), 3.85 (m, 2H), 2.19 (t, 2H), 1.98- 1.90 (m, 1H), 1.78-1.69 (m, 1H), 1.55-1.48 (m, 2H), 1.45 (d, 3H), 1.42-1.28 (m, 4H). [α]_{D}²⁰ = −82°, c = 0.110, chloro- form. |
| 20 | 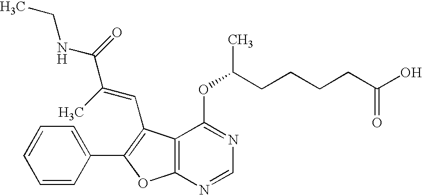 | LC-MS (method 8): Rₜ = 1.88 min; m/z = 452 (M + H)⁺ ¹H-NMR (400 MHz, DMSO-d₆): δ = 11.95 (s, 1H), 8.58 (s, 1H), 8.05 (t, 1H), 7.78 (d, 2H), 7.58- 7.44 (m, 3H), 7.38 (s, 1H), 5.36 (m, 1H), 3.25 (m, 2H), 2.19 (t, 2H), 1.71-1.62 (m, 2H), 1.67 (s, 3H), 1.54-1.46 (m, 2H), 1.42- 1.32 (m, 2H), 1.35 (d, 3H), 1.13 (t, 3H). [α]_{D}²⁰ = −22°, c = 0.085, chloro- form. |
| 21 | 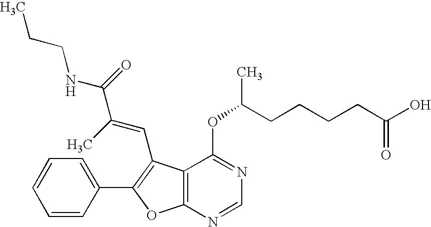 | LC-MS (method 8): Rₜ = 1.99 min; m/z = 466 (M + H)⁺ ¹H-NMR (400 MHz, DMSO-d₆): δ = 11.95 (s, 1H), 8.57 (s, 1H), 8.05 (t, 1H), 7.79 (d, 2H), 7.57- 7.45 (m, 3H), 7.36 (s, 1H), 5.36 (m, 1H), 3.18 (q, 2H), 2.19 (t, 2H), 1.72-1.62 (m, 2H), 1.68 (s, 3H), 1.55-1.45 (m, 4H), 1.40- 1.33 (m, 2H), 1.35 (d, 3H), 0.89 (t, 3H). [α]_{D}²⁰ = −17°, c = 0.075, choro- form. |

### B. Assessment of Pharmacological Efficacy

The pharmacological action of the compounds according to the invention can be demonstrated in the following assays:

B-1. Studies of Binding to Prostacyclin Receptors (IP Receptors) of Human Thrombocyte Membranes

Thrombocyte membranes are obtained by centrifuging 50 ml of human blood (Buffy coats with CDP Stabilizer, from Maco Pharma, Langen) for 20 min at 160×g. Remove the supernatant (platelet-rich plasma, PRP) and then centrifuge again at 2000×g for 10 min at room temperature. Resuspend the sediment in 50 mM tris(hydroxymethyl)amino-methane, which has been adjusted to a pH of 7.4 with 1 N hydrochloric acid, and store at −20° C. overnight. On the next day, centrifuge the suspension at 80 000×g and 4° C. for 30 min. Discard the supernatant. Resuspend the sediment in 50 mM tris(hydroxy-methyl)aminomethane/hydrochloric acid, 0.25 mM ethylene diamine tetraacetic acid (EDTA), pH 7.4, and then centrifuge once again at 80 000×g and 4° C. for 30 min. Take up the membrane sediment in binding buffer (50 mM tris(hydroxymethyl)-aminomethane/hydrochloric acid, 5 mM magnesium chloride, pH 7.4) and store at −70° C. until the binding test.

For the binding test, incubate 3 nM ³H-Iloprost (592 GBq/mmol, from AmershamBioscience) for 60 min with 300-1000 μg/ml of human thrombocyte membranes per charge (max. 0.2 ml) in the presence of the test substances at room temperature. After stopping, add cold binding buffer to the membranes and wash with 0.1% bovine serum albumin. After adding Ultima Gold Scintillator, quantify the radioactivity bound to the membranes using a scintillation counter. The nonspecific binding is defined as radioactivity in the presence of 1 μM Iloprost (from Cayman Chemical, Ann Arbor) and is as a rule <25% of the bound total radioactivity. The binding data (IC₅₀ values) are determined using the program GraphPad Prism Version 3.02.

Representative results for the compounds according to the invention are shown in Table 1:

TABLE 1

| Example No. | IC₅₀ [nM] |
|---|---|
| 15 | 545 |
| 16 | 13 |
| 17 | 1055 |
| 21 | 132 |

B-2. IP-Receptor Stimulation on Whole Cells

The IP-agonistic action of test substances is determined by means of the human erythroleukaemia cell line (HEL), which expresses the IP-receptor endogenously [Murray, R., FEBS Letters 1989, 1: 172-174]. For this, the suspension cells (4×10⁷ cells/ml) are incubated with the particular test substance for 5 minutes at 30° C. in buffer [10 mM HEPES (4-(2-hydroxyethyl)-1-piperazineethanesulfonic acid)/PBS (phosphate-buffered saline, from Oxoid, UK)], 1 mM calcium chloride, 1 mM magnesium chloride, 1 mM IBMX (3-isobutyl-1-methylxanthine), pH 7.4. Next, the reaction is stopped by addition of 4° C. cold ethanol and the charges are stored for a further 30 minutes at 4° C. Then the samples are centrifuged at 10 000×g and 4° C. The resultant supernatant is discarded and the sediment is used for determination of the concentration of cyclic adenosine monophosphate (cAMP) in a commercially available cAMP-radioimmunoassay (from IBL, Hamburg). In this test, IP agonists lead to an increase in cAMP concentration, but IP antagonists have no effect. The effective concentration (EC₅₀ values) is determined using the program GraphPad Prism Version 3.02.

### B-3. Inhibition of Thrombocyte Aggregation in Vitro

Inhibition of thrombocyte aggregation is determined using blood from healthy test subjects of both sexes. Mix 9 parts blood with one part 3.8% sodium citrate solution as coagulant. Centrifuge the blood at 900 rpm for 20 min. Adjust the pH value of the platelet-rich plasma obtained to pH 6.5 with ACD solution (sodium citrate/citric acid/glucose). Then remove the thrombocytes by centrifugation, take them up in buffer and centrifuge again. Take up the thrombocyte deposit in buffer and additionally resuspend with 2 mmol/l calcium chloride.

For the measurements of aggregation, incubate aliquots of the thrombocyte suspension with the test substance for 10 min at 37° C. Next, aggregation is induced by adding ADP and is determined by the turbidimetric method according to Born in an aggregometer at 37° C. [Born G. V. R., J. Physiol. (London) 168, 178-179 (1963)].

### B-4. Measurement of Blood Pressure of Anaesthetized Rats

Anaesthetize male Wistar rats with a body weight of 300-350 g with thiopental (100 mg/kg i.p.). After tracheotomy, catheterize the arteria femoralis for blood pressure measurement. Administer the test substances as solution, orally by oesophageal tube or intravenously via the femoral vein in a suitable vehicle.

### B-5. PAH Model in the Anaesthetized Dog

In this animal model of pulmonary arterial hypertension (PAH), mongrel dogs having a body weight of about 25 kg are used. Narcosis is induced by slow i.v. administration of 25 mg/kg of sodium thiopental (Trapanal®) and 0.15 mg/kg of alcuronium chloride (Alloferin®) and maintained during the experiment by continuous infusion of 0.04 mg/kg/h of Fentanyl®, 0.25 mg/kg/h of droperidol (Dehydrobenzperidol®) and 15 μg/kg/h of alcuronium chloride (Alloferin®). Reflectory effects on the pulse by lowering of the blood pressure are kept to a minimum by autonomous blockage [continuous infusion of atropin (about 10 μg/kg/h) and propranolol (about 20 μg/kg/h)]. After intubation, the animals are ventilated using a ventilator with constant tidal volume such that an end-tidal CO₂ concentration of about 5% is reached. Ventilation takes place with ambient air enriched with about 30% oxygen (normoxia). For measuring the hemodynamic parameters, a liquid-filled catheter is implanted into the femoral artery for measuring the blood pressure. A double-lumen Swan-Ganz® catheter is introduced via the jugular vein into the pulmonary artery (distal lumen for measuring the pulmonary arterial pressure, proximal lumen for measuring the central venous pressure). The left-ventricular pressure is measured following introduction of a micro-tip catheter (Millar® Instruments) via the carotid artery into the left ventricle, and from this, the dP/dt value is derived as a measure for the contractility. Substances are administered i.v. via the femoral vein. The hemodynamic signals are recorded and evaluated using pressure sensors/amplifiers and PONEMAH® as data acquisition software.

To induce acute pulmonary hypertension, the stimulus used is either hypoxia or continuous infusion of thromboxane A₂ or a thromboxane A₂ analog. Acute hypoxia is induced by gradually reducing the oxygen in the ventilation air to about 14%, such that the mPAP increases to values of >25 mm Hg. If the stimulus used is a thromboxane A₂ analog, 0.21-0.32 μg/kg/min of U-46619 [9,11-dideoxy-9a,11α-epoxy-methanoprostaglandin F_{2α}(from Sigma)] is infused to increase the mPAP to >25 mm Hg.

### B-6. PAH Model in Anaesthetized Göttingen Minipig

In this animal model of pulmonary arterial hypertension (PAH), Göttingen minipigs having a body weight of about 25 kg are used. Narcosis is induced by 30 mg/kg of ketamine (Ketavet®) i.m., followed by i.v. administration of 10 mg/kg of sodium thiopental (Trapanal®); during the experiment, it is maintained by inhalation narcosis using enfluran (2-2.5%) in a mixture of ambient air enriched with about 30-35% oxygen/N₂O (1:1.5). For measuring the hemodynamic parameters, a liquid-filled catheter is implanted into the carotid artery for measuring the blood pressure. A double-lumen Swan-Ganz® catheter is introduced via the jugular vein into the pulmonary artery (distal lumen for measuring the pulmonary arterial pressure, proximal lumen for measuring the central venous pressure). The left-ventricular pressure is measured following introduction of a micro-tip catheter (Millar® Instruments) via the carotid artery into the left ventricle, and from this, the dP/dt value is derived as a measure for the contractility. Substances are administered i.v. via the femoral vein. The hemodynamic signals are recorded and evaluated using pressure sensors/amplifiers and PONEMAH® as data acquisition software.

To induce acute pulmonary hypertension, the stimulus used is continuous infusion of a thromboxane A₂ analog. Here, 0.12-0.14 μg/kg/min of U-46619 [9,11-dideoxy-9α,11α-epoxymethanoprostaglandin F_{2α} (from Sigma)] is infused to increase the mPAP to >25 mm Hg.

### C. Exemplary Embodiments of Pharmaceutical Compositions

The compounds of the invention can be converted into pharmaceutical preparations in the following ways:

Tablet:

Composition: p 100 mg of the compound of the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

Production:

The mixture of compound of the invention, lactose and starch is granulated with a 5% strength solution (m/m) of the PVP in water. The granules are mixed with the magnesium stearate for 5 minutes after drying. This mixture is compressed with a conventional tablet press (see above for format of the tablet). A guideline compressive force used for the compression is 15 kN.

Suspension which can be Administered Orally:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

Composition:

500 mg of the compound of the invention, 2.5 g of polysorbate and 97 g of polyethylene glycol 400. 20 g of oral solution correspond to a single dose of 100 mg of the compound according to the invention.

Production:

The compound of the invention is suspended in the mixture of polyethylene glycol and polysorbate with stirring. The stirring process is continued until the compound according to the invention has completely dissolved.

i.v. Solution:

The compound of the invention is dissolved in a concentration below the saturation solubility in a physiologically tolerated solvent (e.g. isotonic saline solution, 5% glucose solution and/or 30% PEG 400 solution). The solution is sterilized by filtration and used to fill sterile and pyrogen-free injection containers.

1. A compound of the formula (I) 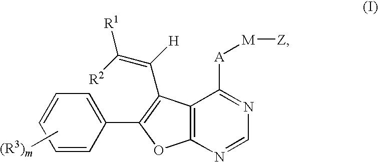
in which
R¹ is (C₁-C₆)-alkyl or a group of the formula —C(═O)—R^{1A} or —CH(OH)—R^{1B} in which R^{1A} represents (C₁-C₆)-alkyl, hydroxyl, (C₁-C₆)-alkoxy, (C₂-C₆)-alkenyloxy, amino, mono-(C₁-C₆)-alkylamino or mono-(C₂-C₆)-alkenylamino and
R^{1B} represents (C₁-C₆)-alkyl,
R² is hydrogen or (C₁-C₄)-alkyl,
R³ is a substituent selected from the group consisting of halogen, cyano, nitro, (C₁-C₆)-alkyl, (C₂-C₆)-alkenyl, (C₂-C₄)-alkynyl, (C₃-C₇)-Cycloalkyl, (C₄-C₇)-Cycloalkenyl, (C₁-C₆)-alkoxy, trifluoromethyl, trifluoromethoxy, (C₁-C₆)-alkylthio, (C₁-C₆)-acyl, amino, mono-(C₁-C₆)-alkylamino, di-(C₁-C₆)-alkylamino and (C₁-C₆)-acylamino,
where (C₁-C₆)-alkyl and (C₁-C₆)-alkoxy for their part may each be substituted by cyano, hydroxyl, (C₁-C₄)-alkoxy, (C₁-C₄)-alkylthio, amino, mono- or di-(C₁-C₄)-alkylamino,
m is the number 0, 1 or 2,
where, if two substituents R³ are present, their meanings may be identical or different,
A is O or N—R⁴, where
R⁴ represents hydrogen, (C₁-C₆)-alkyl, (C₃-C₇)-Cycloalkyl or (C₄-C₇)-Cycloalkenyl,
M is a group of the formula 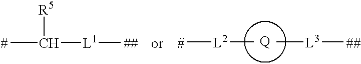
where
# represents the point of attachment to group A and
## represents the point of attachment to group Z,
R⁵ represents hydrogen or (C₁-C₄)-alkyl, which may be substituted by hydroxyl or amino,
L¹ represents (C₁-C₇)-alkanediyl or (C₂-C₇)-alkenediyl which may be mono- or disubstituted by fluorine, or represents a group of the formula *-L^{1A}-V-L^{1B}** in which
* denotes the point of attachment to the group —CHR⁵,
** denotes the point of attachment to group Z,
L^{1A} denotes (C₁-C₅)-alkanediyl which may be mono- or disubstituted by identical or different substituents from the group consisting of (C₁-C₄)-alkyl and (C₁-C₄)-alkoxy,
L^{1B} denotes a bond or (C₁-C₃)-alkanediyl, which may be mono- or disubstituted by fluorine, and
V denotes O or N—R⁶ where
R⁶ represents hydrogen, (C₁-C₆)-alkyl or (C₃-C₇)-Cycloalkyl,
L² represents a bond or (C₁-C₄)-alkanediyl,
L³ represents (C₁-C₄)-alkanediyl which may be mono- or disubstituted by fluorine and in which a methylene group may be replaced by O or N—R⁷, where
R⁷ denotes hydrogen, (C₁-C₆)-alkyl or (C₃-C₇)-Cycloalkyl,
or represents (C₂-C₄)-alkenediyl, and
Q represents (C₃-C₇)-Cycloalkyl, (C₄-C₇)-Cycloalkenyl, phenyl, 5- to 7-membered heterocyclyl or 5- or 6-membered heteroaryl, each of which may be substituted up to two times by identical or different radicals selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino,
where (C₁-C₄)-alkyl for its part may be substituted by hydroxyl, (C₁-C₄)-alkoxy, amino, mono-(C₁-C₄)-alkylamino or di-(C₁-C₄)-alkylamino, and
Z is a group of the formula 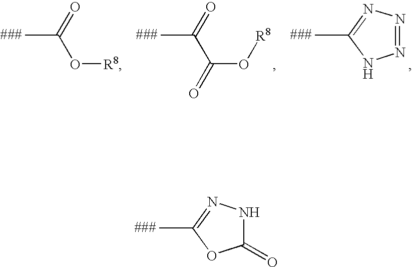
where
### represents the point of attachment to group L¹ or L³ and
R⁸ represents hydrogen or (C₁-C₄)-alkyl,
or a salt thereof.

2. The compound of the formula (I) as claimed in claim 1 in which
R¹ is (C₁-C₄)-alkyl or a group of the formula —C(═O)—R^{1A} in which
R^{1A} represents (C₁-C₄)-alkyl, hydroxyl, (C₁-C₄)-alkoxy, allyloxy, mono-(C₁-C₄)-alkylamino or allylamino,
R² is hydrogen, methyl or ethyl,
R³ is a substituent selected from the group consisting of fluorine, chlorine, cyano, methyl, ethyl, methoxy, ethoxy, trifluoromethyl and trifluoromethoxy,
m is the number 0, 1 or 2,
where, if two substituents R³ are present, their meanings may be identical or different,
A is O or NH,
M is a group of the formula 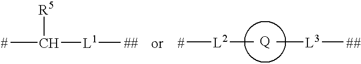
where
# represents the point of attachment to group A and
## represents the point of attachment to group Z,
R⁵ represents hydrogen, methyl or ethyl,
L¹ represents (C₃-C₇)-alkanediyl, (C₃-C₇)-alkenediyl or a group of the formula
*-L^{1A}-V-L^{1B}** in which
* denotes the point of attachment to the group —CHR⁵,
** denotes the point of attachment to group Z,
L^{1A} denotes (C₁-C₃)-alkanediyl which may be mono- or disubstituted by methyl,
L^{1B} denotes (C₁-C₃)-alkanediyl and
V denotes O or N—CH₃,
L² represents a bond, methylene, ethane-1,1-diyl or ethane-1,2-diyl,
L³ represents (C₁-C₃)-alkanediyl or a group of the formula •—W—CH₂—•• or
•—W—CH₂—CH₂—•• in which
• denotes the point of attachment to ring Q,
•• denotes the point of attachment to group Z and
W denotes O or N—R⁷ in which
R⁷ represents hydrogen or (C₁-C₃)-alkyl, and
Q represents cyclobutyl, cyclopentyl, cyclopentenyl, cyclohexyl, pyrrolidinyl, piperidinyl, tetrahydrofuranyl, tetrahydropyranyl, morpholinyl or phenyl, each of which may be substituted up to two times by identical or different radicals selected from the group consisting of fluorine, methyl, ethyl, trifluoromethyl, hydroxyl, methoxy and ethoxy, and
Z is a group of the formula 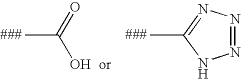
in which
### represents the point of attachment to group L¹ or L³.

3. The compound of the formula (I) as claimed in claim 1 in which
R¹ represents ethyl, n-propyl or a group of the formula —C(═O)—R^{1A} in which
R^{1A} represents ethyl, n-propyl, ethoxy, allyloxy, ethylamino, n-propylamino or allylamino,
R² is hydrogen or methyl,
R³ is fluorine, chlorine or methyl,
m is the number 0 or 1,
A is O or NH,
M is the group of the formula 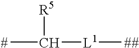
in which
# represents the point of attachment to group A and
## represents the point of attachment to group Z,
R⁵ represents hydrogen or methyl, and
L¹ represents butane-1,4-diyl, pentane-1,5-diyl or a group of the formula *-L^{1A}-O-L^{1B}-** in which
* denotes the point of attachment to the group —CHR⁵,
** denotes the point of attachment to group Z,
L^{1A} denotes methylene or ethane-1,2-diyl which may be mono- or disubstituted by methyl, and
L^{1B} denotes methylene or ethane-1,2-diyl, and
Z represents the group of the formula 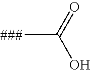
in which
### represents the point of attachment to group L¹.

4. A process for preparing compounds as defined in claim 1 in which Z represents —COON or —C(═O)—COOH, characterized in that a compound of the formula (II) 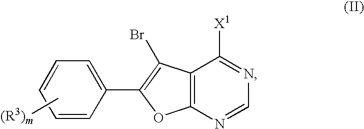
in which R³ and m have the meanings given in any of claims 1 to 3 and
X¹ is a leaving group, such as, for example, halogen, in particular chlorine,
is reacted in an inert solvent in the presence of a base with a compound of the formula (III) 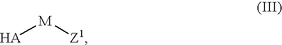
in which A and M have the meanings given in any of claims 1 to 3 and
Z¹ is cyano or a group of the formula —[C(O)]_{y}—COOR^{8A} in which
y represents the number 0 or 1 and
R^{8A} represents (C₁-C₄)-alkyl,
to give a compound of the formula (IV) 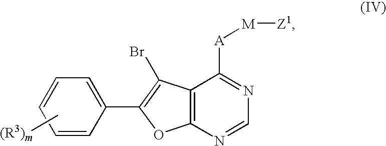
in which A, M, Z¹, R³ and m each have the meanings given above,
which is then either
[A] coupled in an inert solvent in the presence of a base and a suitable palladium catalyst with a boronic acid derivative of the formula (V) or an olefin of the formula (VI) 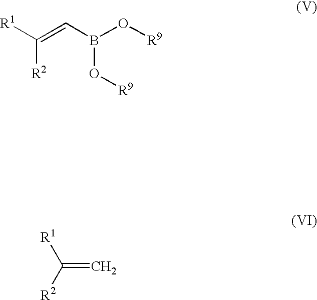
in which R¹ and R² have the meanings given in any of claims 1 to 3 and
R⁹ is hydrogen or (C₁-C₄)-alkyl or both radicals R⁹ together form a —CH₂—CH₂—, —C(CH₃)₂—C(CH₃)₂— or —CH₂—C(CH₃)₂—CH₂— bridge, to give a compound of the formula (VII) 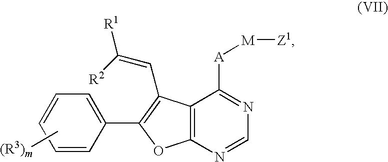
in which A, M, Z¹, R¹, R², R³ and m each have the meanings given above, or
[B] initially converted in an inert solvent in the presence of a base and a suitable palladium catalyst with a vinylboronic acid derivative of the formula (VIII) 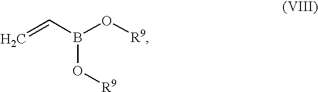
in which R⁹ has the meaning given above
into a compound of the formula (IX) 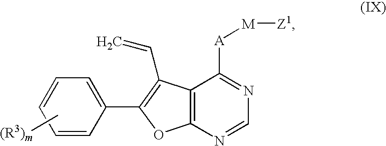
in which A, M, Z¹, R³ and m each have the meanings given above,
then oxidized by reaction with ozone and subsequent treatment with a sulfide to give a compound of the formula (X) 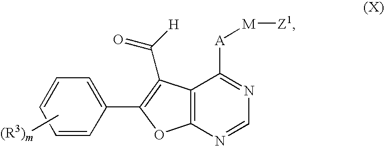
in which A, M, Z¹, R³ and m each have the meanings given above,
and then coupled in an inert solvent in the presence of a base with a phosphorus ylide of the formula (XI) or a phosphonate of the formula (XII) 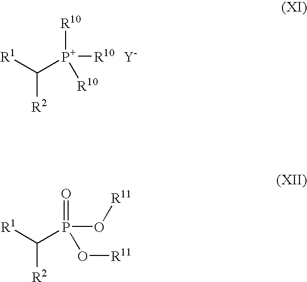
in which R¹ and R² have the meanings given in any of claims 1 to 3 and
R¹⁰ represents phenyl or o-, m- or p-tolyl,
R¹¹ represents (C₁-C₄)-alkyl and
Y⁻ represents a halide anion,
to give a compound of the formula (VII) 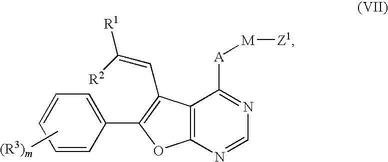
in which A, M, Z¹, R¹, R², R³ and m each have the meanings given above,
and the compounds of the formula (VII) are finally converted by hydrolysis of the ester or cyano group Z¹ into the carboxylic acids of the formula (I-A) 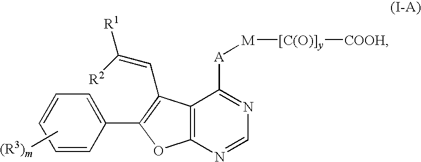
in which A, M, R¹, R², R³, m and y each have the meanings given above,
and these are, if appropriate, reacted with the appropriate (i) solvents and/or (ii) bases or acids to give their solvates, salts and/or solvates of the salts.

5-6. (canceled)

7. A medicament comprising a an inert non-toxic pharmaceutically suitable auxiliary and a compound of the formula (I) 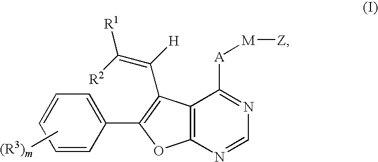
in which
R¹ is (C₁-C₆)-alkyl or a group of the formula —C(═O)—R^{1A} or —CH(OH)—R^{1B} in which R^{1A} represents (C₁-C₆)-alkyl, hydroxyl, (C₁-C₆)-alkoxy, (C₂-C₆)-alkenyloxy, amino, mono-(C₁-C₆)-alkylamino or mono-(C₂-C₆)-alkenylamino and
R^{1B} represents (C₁-C₆)-alkyl,
R² is hydrogen or (C₁-C₄)-alkyl,
R³ is a substituent selected from the group consisting of halogen, cyano, nitro, (C₁-C₆)-alkyl, (C₂-C₆)-alkenyl, (C₂-C₄)-alkynyl, (C₃-C₇)-Cycloalkyl, (C₄-C₇)-Cycloalkenyl, (C₁-C₆)-alkoxy, trifluoromethyl, trifluoromethoxy, (C₁-C₆)-alkylthio, (C₁-C₆)-acyl, amino, mono-(C₁-C₆)-alkylamino, di-(C₁-C₆)-alkylamino and (C₁-C₆)-acylamino, where (C₁-C₆)-alkyl and (C₁-C₆)-alkoxy for their part may each be substituted by cyano, hydroxyl, (C₁-C₄)-alkoxy, (C₁-C₄)-alkylthio, amino, mono- or di-(C₁-C₄)-alkylamino,
m is the number 0, 1 or 2,
where, if two substituents R³ are present, their meanings may be identical or different,
A is O or N—R⁴, where
R⁴ represent hydrogen, (C₁-C₆)-alkyl, (C₃-C₇)-Cycloalkyl or (C₄-C₇)-Cycloalkenyl,
M is a group of the formula 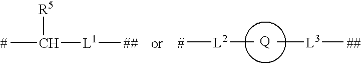
where
# represents the point of attachment to group A and
## represents the point of attachment to group Z,
R⁵ represents hydrogen or (C₁-C₄)-alkyl, which may be substituted by hydroxyl or amino,
L¹ represents (C₁-C₇)-alkanediyl or (C₂-C₇)-alkenediyl which may be mono- or disubstituted by fluorine, or represents a group of the formula *-L^{1A}-V-L^{1B}-** in which
* denotes the point of attachment to the group —CHR⁵,
** denotes the point of attachment to group Z,
L^{1A} denotes (C₁-C₅)-alkanediyl which may be mono- or disubstituted by identical or different substituents from the group consisting of (C₁-C₄)-alkyl and (C₁-C₄)-alkoxy,
L^{1B} denotes a bond or (C₁-C₃)-alkanediyl which may be mono- or disubstituted by fluorine, and
V denotes O or N—R⁶ where
R⁶ represents hydrogen, (C₁-C₆)-alkyl or (C₃-C₇)-Cycloalkyl,
L² represents a bond or (C₁-C₄)-alkanediyl,
L³ represents (C₁-C₄)-alkanediyl which may be mono- or disubstituted by fluorine and in which a methylene group may be replaced by O or N—R⁷, where
R⁷ denotes hydrogen, (C₁-C₆)-alkyl or (C₃-C₇)-Cycloalkyl,
or represents (C₂-C₄)-alkenediyl, and
Q represents (C₃-C₇)-Cycloalkyl, (C₄-C₇)-Cycloalkenyl, phenyl, 5- to 7-membered heterocyclyl or 5- or 6-membered heteroaryl, each of which may be substituted up to two times by identical or different radicals selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino, where (C₁-C₄)-alkyl for its part may be substituted by hydroxyl, (C₁-C₄)-alkoxy, amino, mono-(C₁-C₄)-alkylamino or di-(C₁-C₄)-alkylamino, and
Z is a group of the formula 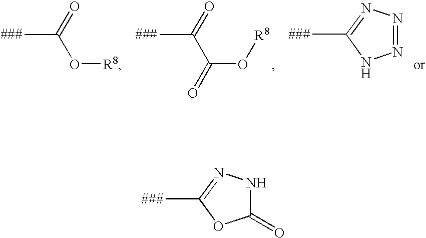
where
### represents the point of attachment to group L¹ or L³ and
R⁸ represents hydrogen or (C₁-C₄)-alkyl,
or a salt thereof.

8-9. (canceled)

10. A method for the treatment and/or prophylaxis of angina pectoris, pulmonary hypertension, thromboembolic disorders and peripheral occlusive diseases in humans and animals using an effective amount of at least one compound as defined in claim 1.
